SEQUENCE LISTING (44 sequences)

SEQ ID NO: 1 - DNA, 19 bp, Artificial
  Synthetic oligonucleotide
acgggttatttttttttcg

SEQ ID NO: 2 - DNA, 20 bp, Artificial
  Synthetic oligonucleotide
taaaatatacgccctccacg

SEQ ID NO: 3 - DNA, 19 bp, Artificial
  Synthetic oligonucleotide
atgggttattttttttttg

SEQ ID NO: 4 - DNA, 20 bp, Artificial
  Synthetic oligonucleotide
taaaatatacaccctccaca

SEQ ID NO: 5 - DNA, 20 bp, Artificial
  Synthetic oligonucleotide
tgattgtcgttcgtagtacg

SEQ ID NO: 6 - DNA, 20 bp, Artificial
  Synthetic oligonucleotide
cgacaatttaataacacgcg

SEQ ID NO: 7 - DNA, 20 bp, Artificial
  Synthetic oligonucleotide
tgattgttgtttgtagtatg

SEQ ID NO: 8 - DNA, 20 bp, Artificial
  Synthetic oligonucleotide
caacaatttaataacacaca

SEQ ID NO: 9 - DNA, 25 bp, Artificial
  Synthetic oligonucleotide
ggttttgttagggataggatttttt

SEQ ID NO: 10 - DNA, 25 bp, Artificial
  Synthetic oligonucleotide
actacctccaacaccccttataact

SEQ ID NO: 11 - DNA, 21 bp, Artificial
  Synthetic oligonucleotide
agtatggagttagtagaagtt

SEQ ID NO: 12 - DNA, 25 bp, Artificial
  Synthetic oligonucleotide
tcacaagtccaacacttaaataact

SEQ ID NO: 13 - DNA, 20 bp, Artificial
  Synthetic oligonucleotide
tagtgagggggcagtggaac

SEQ ID NO: 14 - DNA, 20 bp, Artificial
  Synthetic oligonucleotide
aggagggggtttcgggaata

SEQ ID NO: 15 - DNA, 24 bp, Artificial
  Synthetic oligonucleotide
caaaagagccgctgaagggaaaca

SEQ ID NO: 16 - DNA, 24 bp, Artificial
  Synthetic oligonucleotide
ttcttcagcttccgggctcccaga

SEQ ID NO: 17 - DNA, 20 bp, Artificial
  Synthetic oligonucleotide
cctcggacaccttgcctgaa

SEQ ID NO: 18 - DNA, 21 bp, Artificial
  Synthetic oligonucleotide
cgccaacagagtgtccaagac

SEQ ID NO: 19 - DNA, 20 bp, Artificial
  Synthetic oligonucleotide
tagtgagggggcagtggaac

SEQ ID NO: 20 - DNA, 20 bp, Artificial
  Synthetic oligonucleotide
aggagggggtttcgggaata

SEQ ID NO: 21 - DNA, 21 bp, Artificial
  Synthetic oligonucleotide
gatggccaattctgccataag

SEQ ID NO: 22 - DNA, 22 bp, Artificial
  Synthetic oligonucleotide
gtctggttcatccccattgact

SEQ ID NO: 23 - DNA, 21 bp, Artificial
  Synthetic oligonucleotide
acacctatggaattgtcctgc

SEQ ID NO: 24 - DNA, 22 bp, Artificial
  Synthetic oligonucleotide
gaccagagagagctcagatacg

SEQ ID NO: 25 - DNA, 170 bp, Artificial
  Synthetic oligonucleotide
agtacggagccagcagaagtccgacccttcctgggaatgggctgtaccgagaggtccgactagccccagggttttagtgagggggcagtggaactcagcgagggactgagagcttcacagcatgcacgagtttgatgccagagaaaaagtcggaagtgccggacttgtga

SEQ ID NO: 26 - DNA, 24 bp, Artificial
  Synthetic oligonucleotide
gtttttatagttttcggttttcgg

SEQ ID NO: 27 - DNA, 24 bp, Artificial
  Synthetic oligonucleotide
actcccaatttaatttcgtaaacg

SEQ ID NO: 28 - DNA, 23 bp, Artificial
  Synthetic oligonucleotide
tttttatagtttttggtttttgg

SEQ ID NO: 29 - DNA, 26 bp, Artificial
  Synthetic oligonucleotide
cactcccaatttaatttcataaacac

SEQ ID NO: 30 - DNA, 25 bp, Artificial
  Synthetic oligonucleotide
ataagtttattaatcggcgaagttc

SEQ ID NO: 31 - DNA, 25 bp, Artificial
  Synthetic oligonucleotide
aataaaaaaaacaaaaaaaaccgat

SEQ ID NO: 32 - DNA, 25 bp, Artificial
  Synthetic oligonucleotide
aagtttattaattggtgaagtttga

SEQ ID NO: 33 - DNA, 25 bp, Artificial
  Synthetic oligonucleotide
ccaataaaaaaaacaaaaaaaacca

SEQ ID NO: 34 - DNA, 23 bp, Artificial
  Synthetic oligonucleotide
gttggggtagtttttatagtttt

SEQ ID NO: 35 - DNA, 30 bp, Artificial
  Synthetic oligonucleotide
ataaccctttctaactaacatttcttatac

SEQ ID NO: 36 - DNA, 24 bp, Artificial
  Synthetic oligonucleotide
ggaggatttagagttttggttttt

SEQ ID NO: 37 - DNA, 25 bp, Artificial
  Synthetic oligonucleotide
aacccttctctaactctcaactcaa

SEQ ID NO: 38 - DNA, 25 bp, Artificial
  Synthetic oligonucleotide
tgtgatttgggatattttttttatg

SEQ ID NO: 39 - DNA, 25 bp, Artificial
  Synthetic oligonucleotide
aaaactccttctccaactccaatat

SEQ ID NO: 40 - DNA, 21 bp, Artificial
  Synthetic oligonucleotide
gcccttccgagagcagcaaag

SEQ ID NO: 41 - DNA, 25 bp, Artificial
  Synthetic oligonucleotide
aggtggacgctgcggctaatctcta

SEQ ID NO: 42 - DNA, 266 bp, Homo sapiens
  misc_feature: F1 sequence of HOXA 10 gene
gttggggcagcccccacagctcccggccttcgggccaaggtgtcggggtgcgtctcctggcccatcaatacagattacatatttatatcaatcgcgggctctgagggcgccctcggagagcggccccgcgcctacgaaaccaaactgggagtggtcgcgcggaaactctggctcgggattggctgcgggcgcccgccgcggtgcggggggattgctaatcgtattcagcatgttttgcacaagaaatgtcagccagaaagggctat

SEQ ID NO: 43 - DNA, 291 bp, Homo sapiens
  misc_feature: F2 sequence of HOXA 10 gene
ggaggacccagagccctggtccctgcccagcctgcgcggcgcggcccacgcggggggagggggagggagggaaagtagctcgcccgcagatagcgcggatgtttgtaaggcatccaaaataagcagccgccagcgccaataaataagcccattaaccggcgaagttcgagtgtacgatcccccatgcttttttcaaagttgctgaggggcgggaatcttcgtggcgggaagaagaaaaggcaaatccggcctggaagcggggggccctgagctgagagccagagaagggcc

SEQ ID NO: 44 - DNA, 216 bp, Homo sapiens
  misc_feature: F3 sequence of HOXA 10 gene
tgtgacttgggacatctctcttatgggagaaacggaggcaattcacccccgcgggcagcccgtgtggcctcgacttaatcatcccctctttattctcttacatgccaggcaattccaaaggtgaaaacgcagccaactggctcacggcaaagagtggtcggaagaagcgctgcccctacacgaagcaccagacactggagctggagaaggagtttc

(19) United States
(12) Patent Application Publication
(10) Pub. No.: US 2007/0287676 A1
(43) Pub. Date: Dec. 13, 2007

(54) Diagnosis and treatment of endometriosis

(76) Inventors: Sun-Wei Guo, Shanghai (CN); Yan Wu, West Allis, WI (US)

Correspondence Address: QUARLES & BRADY LLP, 33 E. MAIN ST, SUITE 900, P.O BOX 2113, MADISON, WI, 53701-2113, US

(21) Appl. No.: 11/803,993

(22) Filed: May 16, 2007

Related U.S. Application Data
(60) Provisional application No. 60/800,873, filed on May 16, 2006.

(51) Int. Cl. C12Q 1/68; A61K 31/165; A61K 31/19; A61K 31/70; C07H 21/04; G01N 33/53; C07K 16/00; C12N 9/10; C12Q 1/48

(52) U.S. Cl. 514/43; 435/15; 435/193; 435/6; 435/7.92; 514/557; 514/619; 530/387.1; 536/22.1; 536/24.33

(57) ABSTRACT

Disclosed here are simple, non-invasive screening methods for diagnosing endometriosis, in particular, the type that is resistant to conventional progestin and progesterone therapy. Biomarkers for identifying subjects with endometriosis are disclosed. Alternative therapeutic methods with reduced side effects and compositions for treating endometriosis are also disclosed.

### CROSS-REFERENCE TO RELATED APPLICATIONS

This application claims the benefit of U.S. Provisional Application No. 60/800,873 filed May 16, 2006. The application is incorporated herein by reference in its entirety.

### STATEMENT REGARDING FEDERALLY SPONSORED RESEARCH OR DEVELOPMENT

Not applicable.

### BACKGROUND OF THE INVENTION

Endometriosis, defined as the ectopic presence of endometrial glandular and stromal cells outside the uterine cavity, is a common benign gynecological disorder. It is a leading cause of disability in women of reproductive age, resulting in dysmenorrhea, pelvic pain, and subfertility. Current treatment modalities include medical, surgical, or a combination of both. Today's surgical treatment of endometriosis is performed mostly by laparoscopy. However, the recurrence risk after surgery is high: 7-30% of patients reported recurrences three years after laparoscopic surgery. The risk increases to 40-50% five years after the treatment. In fact, recurrent symptoms occur in about 10% of women even after hysterectomy and bilateral salphingo-oophorectomy are performed. Because of this high recurrence risk, continued medical treatment is required.

Since estrogen is a potent mitogen for ectopic implants, the current medical treatment for endometriosis has so far focused on the hormonal alteration of the menstrual cycle with a major goal to produce a pseudo-pregnancy, pseudo-menopause, or chronic anovulation, creating an acyclic, hypoestrogenic environment. This is achieved either by blocking ovarian estrogen secretion (GnRH agonists (GnRH-a)), by inducing pseudo-pregnancy (progestins), or by locally inhibiting estrogenic stimulation of the ectopic endometrium (progestins, androgenic progestins).

All current major therapies are effective in treating pains, most likely through suppression of proliferation of the implants and reduction of adhesion formation. The relief of pain, however, appears to be relatively short-term. In addition, about 9% of women with endometriosis simply do not respond to progestin treatment, and its cause is largely unknown. In fact, investigations have noted progesterone resistance within eutopic and ectopic endometrium of women with endometriosis. Moreover, all of the therapies known to date have many undesirable, and sometimes severe, side effects. Even the most recent therapies for treating endometriosis, the aromatase inhibitors, have side effects, which include bone loss. For example, in premanopausal endometriosis, estrogen depletion due to suppression of aromatase activity in the hypothalamus causes FSH secretion and ovarian stimulation. In this case an aromatase inhibitor is administered along with a GnRH-a, a progestin, progesterone, or a combination, which results in multiple side effects. Clearly, there is pressing need for a simple and rapid test for predicting progesterone resistance in women and employing therapies for and treating endometriosis with less and/or milder side effects.

### BRIEF SUMMARY OF THE INVENTION

The present application is summarized as novel methods of diagnosing and treating endometriosis. Specifically, this application provides simple non-invasive epigenetic screening methods and biomarkers for identifying a subject with endometriosis, in particular the type that is resistant to progestin and progesterone. Such screening tools enable clinicians to determine if a subject with endometriosis is a suitable candidate for conventional progestin/progesterone therapy. Also disclosed are alternative methods and compositions for more effectively treating and ameliorating the symptoms of endometriosis with fewer and milder side effects than currently available therapies.

In one aspect, a method is disclosed for diagnosing endometriosis in an subject. The method includes a) assaying a biological sample obtained from the subject to determine the expression pattern of any of the genes comprising progesterone receptor-B (PR-B), HOXA10, and DNA methyltransferase (DNMT), or a combination thereof in the sample; b) comparing the expression patterns of the PR-B, HOXA10 and DNMT genes of the subject with that of a non-endometriotic subject; and c) diagnosing the subject as having endometriosis if the expression of any one of the selected genes is altered as compared to the non-endometriotic subject.

One aspect of the application provides a screening method to determine if a subject has endometriosis that is resistant to progestin and/or progesterone therapy. This diagnostic method includes includes (a) providing a biological sample of the subject to be screened; and (b) assaying the sample for the presence of a biomarker associated with endometrial pathology. A suitable biomarker may include hypermethylation of a progesterone receptor promoter, such as for example a hypermethylated progesterone receptor promoter at isoform B (PR-B) and aberrant methylation of the HOXA10 gene.

A DNA screening assay is a suitable assay for detecting hypermethylation of the PR-B promoter and/or aberrant methylation of the HOXA10 gene relative to a control. Such an assay may include, but is not limited to hybridization, sequencing, microarray, quantitative PCR, and other suitable assays known in the art. Notably, hypermethylation can be detected by methylation analysis, suitably methylation specific PCR and bisulfite sequencing, and other methylation detection assays.

A related aspect of the application provides a screening method to determine if a subject has endometriosis that is resistant to progestin and/or progesterone therapy. This diagnostic method includes (a) providing a biological sample of the subject to be screened; and (b) assaying the biological sample for the presence of a transcriptional, epigenetic or proteomic biomarker associated with endometrial pathology. A suitable biomarker includes an increased expression level of DNA methyltransferase (DNMT), such as: DNMT1, DNMT3A and DNMT3B relative to a control. Suitable assays for detecting the increased expression of DNMT include, but are noted limited to RNA screening assays (e.g., quantitative RT-PCR of mRNA expression) and immunoassays (e.g., immunofluorescence staining).

The disclosed diagnostic methods and biomarkers can be used to monitor disease progression, treatment efficacy, and relapse of endometriosis.

In one aspect, a method is disclosed for selecting a therapy to treat endometriosis including (a) assaying a biological sample, such as menstrual fluid, for the presence of at least one biomarker associated with endometrial pathology, wherein the biomarker is (i) a hypermethylation of PR-B promoter, (ii) aberrant methylation of the HOXA10 gene, and/or (iii) an increase in DNA methyltransferase expression level in such cells relative to a control; (b) identifying endometriotic cells having the biomarker; and (c) selecting a patient-specific therapy according to the results of the detection. The therapy may include histone deacetylase inhibitors (HDACIs) alone or in conjunction with demethylation agents, or retinoic acid or other suitable therapeutic compounds described herein.

In another aspect, biomarkers associated with and/or predictive of endometriosis are disclosed. The biomarkers includes (i) an increased expression level of a DNA methyltransferase (DNMT), suitably DNMT1, DNMT3A, and DNMT3B; (2) hypermethylated progesterone receptor promoter, suitably isoform B; (3) aberrant methylation of the HOXA10 gene in a subject with endometriosis relative to a control.

In another aspect, a method is disclosed for restoring normal expression of DNA methyltransferases (DNMT) in a subject having endometriosis. The method including administering an effective amount of a histone deacetylase inhibitor (HDACI), such as valproic acid, alone or in combination with a demethylation agent (suitably, 5-aza-2′-deoxycytidine (decitabine), hydralazine, or 5-azacytidine) or retinoic acid to the subject. Suitable HDACIs include valproic acid or suberoylanilide hydroxamic acid. The administration of HDACIs results in upregulation of progesterone receptor gene through (i) a decreased progesterone receptor (PR) gene methylation, (ii) an increased progesterone binding, and (iii) an increased responsiveness to progesterone therapy.

In another aspect, a method is disclosed for treating a subject having endometriosis by administering to the subject a therapeutically effective amount of histone deacetylase inhibitors alone or in combination with a demethylation agent sufficient to treat endometriosis. The combination of the inhibitor(s) and agent has a synergistic effect in suppressing growth and inflammation of endometriotic cells, resulting in the effective therapeutic treatment of endometriosis.

In this aspect, a suitable demethylation agent includes 5-azacytidine, 5-aza-2′-deoxycytidine, or hydralazine, and suitable HDACIs include suberoylanilide hydroxamic acid, SAHA, or valproic acid, VPA. A suitable retinoic acid is all-trans retinoic acid.

In another aspect, a method is disclosed for treating a subject having endometriosis by administering to the subject a therapeutically effective amount of retinoic acid alone or in combination with histone deacetylase inhibitor, suitably valproic acid, thereby resulting in the effective therapeutic treatment of the endometriosis.

In another aspect, a kit is provided for assaying biological materials for the presence of promoter hypermethylation of progesterone receptor isoform B (PR-B) or aberrant methylation of HOXA10 gene relative to a control. The kit includes, but is not limited to at least one primer pair capable of selectively hybridizing to the progesterone receptor isoform B (PR-B) gene. The kit includes, but is not limited to at least one primer pair capable of selectively hybridizing to the HOXA10 gene.

In another aspect, a kit is provided for assaying a biological material for the presence of an increased DNA methyltransferase expression level in a biological sample relative to a control. The kit may include at least one primer pair or one oligonucleotide capable of selectively hybridizing to the DNA methyltransferase (DNMT) genes, where in the DNMTs include DNMT1, DNMT3A, and DNMT3B.

In another aspect, a kit for detecting endometriosis is provided, wherein the kit includes an antibody against a DNA methyltransferase (DNMT), suitably DNMT1, DNMT3A, and DNMT3B. The detection is performed by an immunoassay or by quantitative PCR.

Unless otherwise defined, all technical and scientific terms used herein have the same meaning as commonly understood by one of ordinary skill in the art to which this invention belongs. Although suitable methods and materials for the practice or testing of the present invention are described below, other methods and materials similar or equivalent to those described herein, which are well known in the art, can also be used.

Other objects, advantages and features of the present invention will become apparent from the following specification taken in conjunction with the accompanying drawings.

### BRIEF DESCRIPTION OF THE SEVERAL VIEWS OF THE DRAWINGS

FIG. 1 shows a genomic structure of the human progesterone receptor gene. The gene has CpG islands in the promoter region specific for isoform A (PR-A) and isoform B (PR-B). The fragment F1 of PR-A (100 bps) and fragment F2 of PR-B (200 bps), located in the 1621-1639 bp/1700-1719 bp and 867-886 bp/1047-1066 bp at 5′-end of exon 1, respectively, were PCR amplified after bisulfite conversion, and then directly sequenced using the ABI 3700 DNA Sequencer. ATG1 and ATG2 are transcription start sites for PR-A and PR-B, respectively, which are located in 1455 bp and 1947 bp at 5′-end of exon 1, respectively.

FIG. 2 is a tabular list of clinical characteristics of 16 patients, along with the availability of their tissue samples for bisulfite sequencing and mRNA analysis.

FIG. 3 shows partial methylation at PR-B promotor by MSP with bisulfite-treated ectopic and eutopic endometrial epithelial cells from endometriosis patients and normal controls. Sample U shows reactions using the PR primer set specific for the unmethylated CpG sites giving rise to a 200 bp band. Sample M shows reactions using the primer set specific for the methylated CpG sites giving rise to 200 bp band. Lanes 1-8 were treated DNA from normal controls, while lanes 9-18 were those from endometrium of patients with endometriosis. Lanes 19-34 were treated DNA from ectopic endometrial tissues (lesion). Lane 35 was a negative control (H₂O). Three lanes shown intensive multiple bands are markers. The result showed that the promoter region of PR-B from endometriosis patients was partially methylated as compared with the controls.

FIGS. 4A-B show the results of bisulfite sequencing of the promoter region of PR-B containing 9 CpG sites. (A) is the partial nucleotide sequence of PGR-B promoter (SEQ ID NO: 25). (B) The result demonstrated methylation levels of endometriotic and endometrial samples from 8 patients, and 4 healthy control individuals. Gray scale shown represents the level of methylation for each CpG site.

FIG. 5 shows a boxplot of PR-B expression in laser capture microdissection harvested epithelial cells in different tissue groups.

FIGS. 6A-B show semi-quantification by RT-PCR of mRNA expression of DNMT1, DNMT3A and DNMT3B in six pairs of ectopic and eutopic endometrial epithelium in 6 control samples. (A) Lane 1-6: ectopic endometrial epithelium; Lane 7-12: eutopic endometrial epithelium; Lane 13-18, normal endometrial epithelium from endometriosis-free women. Lane 19, no template as negative control for PCR. GDPAH served as endogenous control. (B) Expression levels of DNMTs relative to GAPDH in 18 tissue samples.

FIGS. 7A-B show median gene expression levels in the ectopic and eutopic endometrium of women with endometriosis and the control group. The expression levels were normalized by (A) PCNA (46) and (B) GAPDH. The p-values listed in the figure represent results of randomization test.

FIGS. 8A-F show expression of DNMT1 examined by immunofluorence staining. The ectopic epithelium showed stronger DNMT1 immunoreactivity than eutopic epithelium and normal epithelium from tubal ligation women. Cytokaratin (8/18) was used to distinguish epithelium structures in endometrial glands. Original magnification: X20.

FIG. 9 shows the transcript structure of human HOXA 10 gene. This gene has several CpG islands within 5′ upstream region of exon1 and intron region. Three fragments within CpG islands (F1, F2 and F3) were sequenced after bisulfite treatment.

FIGS. 10A-C show partial methylation in the F2 fragment of HOXA10 by MSP with bisulfite-treated endometrial tissue samples from endometriosis patients, normal controls and normal lymphocytes. Sample U shows reactions using the HOXA10 primer set specific for the unmethylated CpG sites giving rise to a 260-bp band. Sample M shows reactions using the primer set specific for the methylated CpG sites giving rise to a 260-bp band. WT, unmodified/wild type. Mt is a molecular marker. (A). Lanes 1-12 were bisulfite treated DNA from patients with endometriosis, while lanes 13-18 were those from normal controls. Notice the very faint bands in the M lanes of all endometriosis patients and two controls, suggesting partial methylation. (B). Lane 1-10 were bisulfite treated DNA from menstrual debris of healthy women. Notice the absence of the dark band in the M lane in all controls. (C). Lanes 1 and 2 were treated DNA from normal lymphocytes. Lane 3-6 were methylation positive control. Lane 7 was DNA from normal lymphocytes and PCR amplified using wild type primer for monitoring bisulfite treatment. Lane 8 was a negative control (H2O). The results showed that the F2 fragment of HOXA10 gene from endometriosis patients was partially methylated as compared with the controls.

FIGS. 11A-E show bisulfite sequencing of HOXA 10 F3 fragment using bisulfite modified genomic DNA from endometrium of endometriosis patients (panel A and B), normal control woman (panel C), and normal blood lymphocytes (panel D), and normal genomic DNA treated with methylase SssI as 100% methylation positive control (panel E). Methylated CpG sites (red arrows indicated). Unmethylated CpG sites (black arrows indicated).

FIGS. 12A-B show that F1 fragment containing 22 CpG sites started from −25 bp to −300 bp 5′upstream of exon1. The result demonstrated methylation levels of endometrium samples from 6 endometriosis patients and 9 healthy control individuals. Gray scale shown at the right represents the level of methylation for each CpG site.

FIGS. 13A-B show bisufite sequencing of F2 containing 21 CpG sites. The result demonstrated methylation levels of endometrium samples from 6 endometriosis patients and 6 healthy control individuals. Gray scale shown at the right represents the level of methylation for each CpG site.

FIGS. 14A-B show bisufite sequencing of F3 fragment containing 10 CpG sites. The result demonstrated methylation levels of endometrium samples from 6 endometriosis patients and 8 healthy control individuals. Gray scale shown at the right represents the level of methylation for each CpG site.

FIG. 15 is a table showing real-time RT-PCR quantitative amounts of HOXA10 and 18S mRNA in endometrium samples. Ct: Cycle number at which RNA reached a threshold amount of fluorescence above background. ES: early secretory phase; EP: early proliferative phase; LP: late proliferative phase. P1,3,4,5=patients with endometriosis, C2 and C3=controls without endometriosis.

FIG. 16 is a tablular list of primers used in the real-time RT-PCR analysis.

FIG. 17 is a tablular list of characteristics of women with endometriosis and of controls.

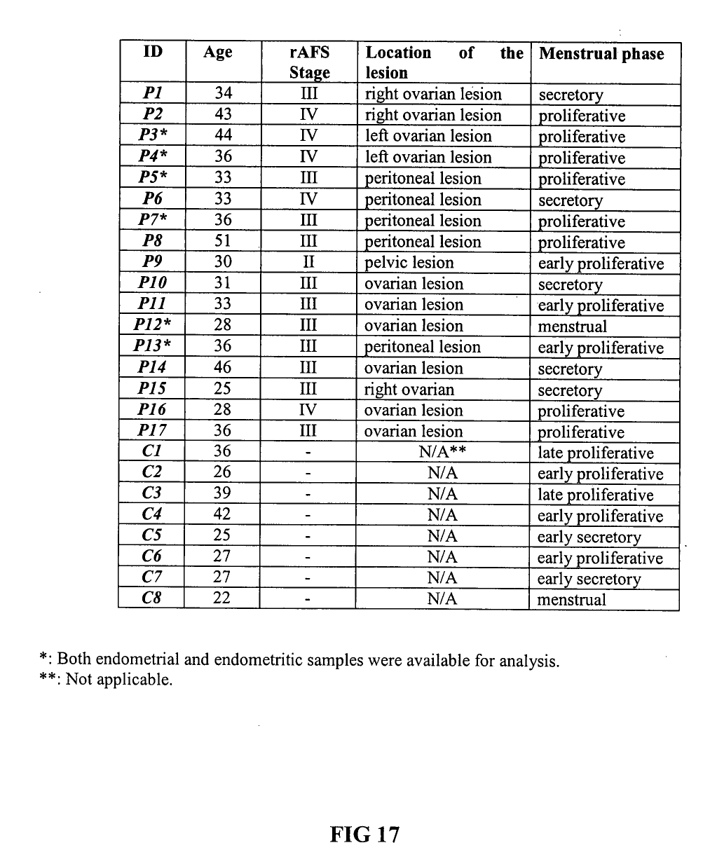

FIG. 18 shows a Western blot analysis of the expression of ERα and ERβ in the Yale human endometrial stromal (YHES) cells. The YHES cells were treated, respectively, with TSA (10 μM), CDB (100 μM), NAC (10 mM), ICI (100 μM) and ethanol alone. Cell lysates were prepared after 24 hr of treatment.

FIG. 19 shows the effects of various compounds on endometrial stromal cells proliferation. The YHES cells were cultured for 48 hours under the physical concentration of E2 and P (10-7M), followed by treatment of H₂O₂, TSA, CDB, RU486, ICI, NAC, or no treatment (vehicle alone as control). Concentrations used were indicated. Cell proliferation was measured by MTT assays. Cell growth was expressed as relative absorbance fold change to control. The results represent three independent experiments. * indicates P<0.05, and ** indicates P<0.01.

FIG. 20 shows the effects of various compounds on H2O2-induced cell proliferation. The YHES cells were pretreated with or without H₂O₂ (1 μM) for 48 hours under the physical condition of E₂ (10-9M) and progesterone (10-7M), and the culture medium was replaced with medium containing TSA (100 μM), ICI (100 μM), RU486 (100 μM), CDB (100 μM), NAC (10 mM) and ethanol alone, respectively. MTT assay was performed after 48 hours of treatment. The results represent three independent experiments. * indicates significantly different from the control (P<0.01).

FIGS. 21A-F show the morphology of YHES cells after 24 hr, 48 hr, and 4 weeks of treatment with vehicle (control), H₂O₂, TSA, RU486, CDB, and NAC. The micrographs of the first two rows were taken using 20× magnification, and those of the third row were taken using 10× magnification. The micrographs of the first row and the first two micrographs of second row were obtained using differential inference contrast (DIC) optic; the others were obtained without the DIC optic.

FIG. 22 shows expression of PR-A, PR-B, Fas, FasL and AR in YHES cells treated with TSA, CDB and NAC. Total RNA was isolated from cells treated with TSA (10 μM), CDB (100 μM), NAC (10 mM) and ethanol alone, at 8 hr, 18 hr and 36 hr after treatment, respectively. RT-PCR was performed to measure mRNA levels of each gene. 18s served as endogenous control. Note the scale of the fold change in FasL expression. The dotted line represents fold change of 1, i.e., no difference from the control group.

FIGS. 23A-E shows a comparison of AR, PR-A, PR-B, Fas, and FasL expression in treated cells versus untreated cells. mRNA levels were measured by real-time RT-PCR. The expression value of each treatment group, measured in triplicate, was represented as relative expression after normalized to the untreated control cells.

FIG. 24 shows a Western blot analysis of protein level of AR on treated YHES cells. YHES cells were treated with TSA (10 μM), CDB (100 μM), NAC (10 mM) and ethanol alone. Cell lysates were prepared after 16 hr of treatment.

FIG. 25 shows the effects of TSA on proliferation of endometriotic cells. tRA is all-trans retinoic acid.

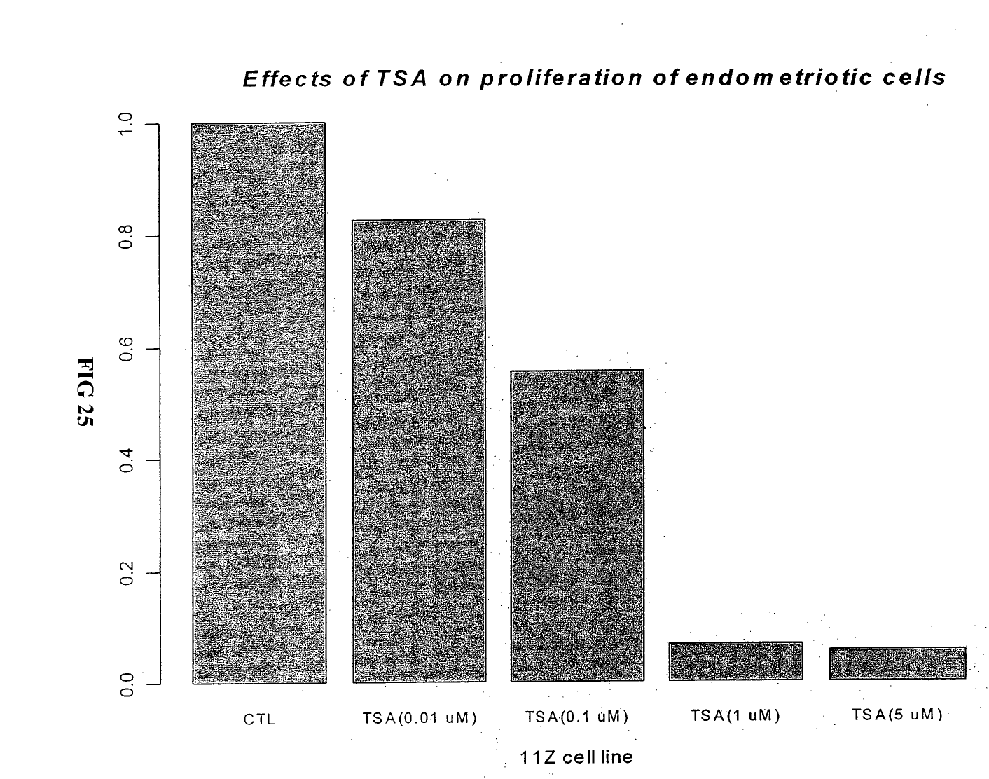

FIG. 26 shows the effects of TSA and tRA on proliferation of endometriotic cells.

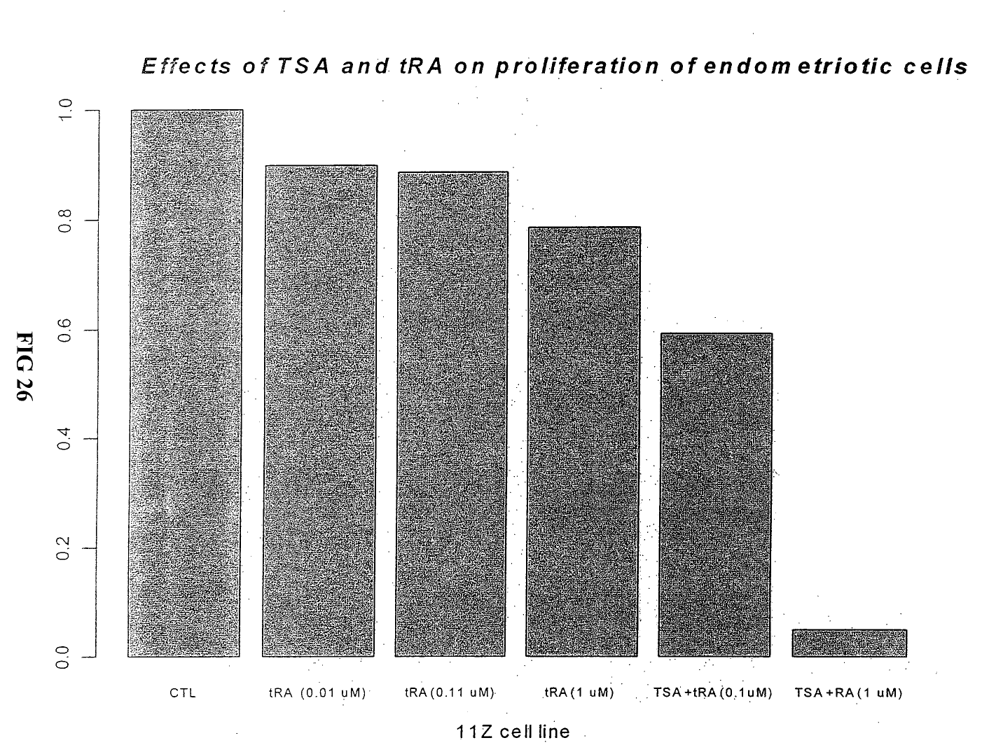

FIG. 27 shows the effects of TSA and 5-Aza on proliferation of endometriotic cells. 5-aza is 5-azacytidine, a demethylation agent.

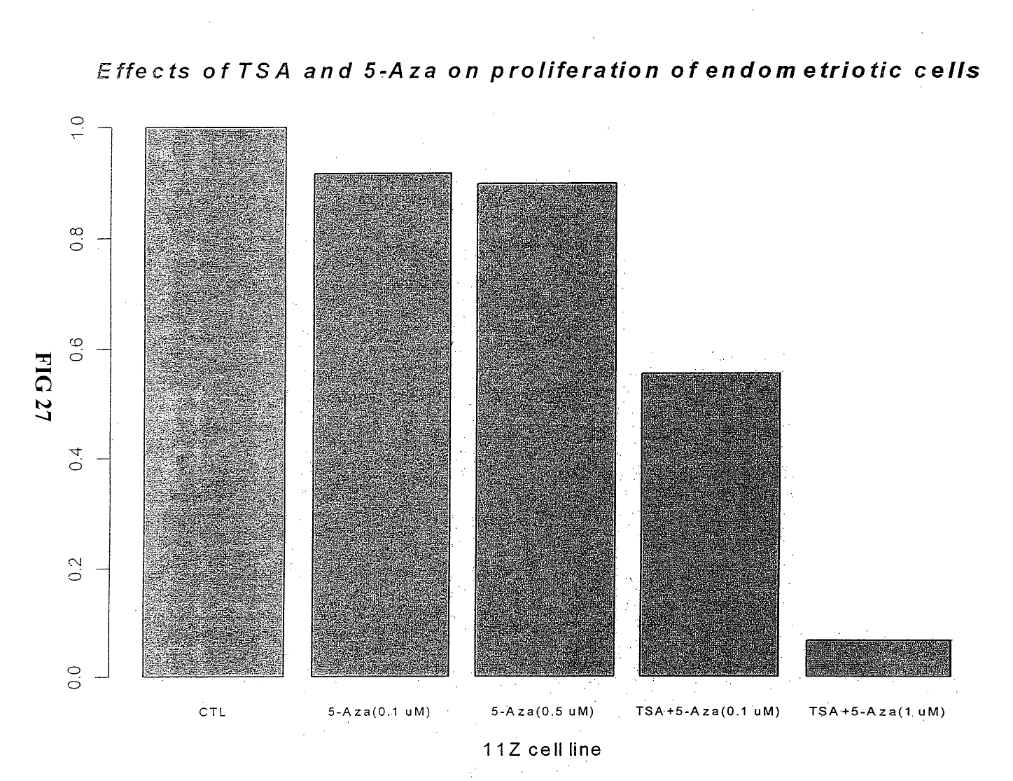

FIG. 28 is table showing patient characteristics, physical and ultrasound evaluation, and outcome.

### DETAILED DESCRIPTION OF THE INVENTION

The present application relates to novel methods of diagnosing and treating endometriosis. Specifically, this application provides simple non-invasive epigenetic screening methods and biomarkers for identifying a subject with endometriosis, in particular the type that is resistant to progestin and progesterone. Such screening tools enable clinicians to determine if a subject with endometriosis is a suitable candidate for conventional progestin/progesterone therapy. Also disclosed are alternative methods and compositions for more effectively treating and ameliorating the symptoms of endometriosis with fewer and milder side effects than currently available therapies.

Applicants discovered that in a majority of women with endometriosis, the promoter regions of certain genes were aberrantly methylated. This resulted in resistance to progestin/progesterone therapy. More specifically applicants identified that there were regions in the PR-B and HOXA10 genes that were abberently methylated, which effectively silences these genes. Applicants then linked this hypermethylation with endometriosis that was resistant to progestin/progesterone therapy.

Accordingly, applicants determined that the methylation state of nucleic acids of certain genes, particularly regulatory sequences, is diagnostic of endometriosis. More particularly, the hypermethylation of certain nucleotides localized in CpG islands has been shown to affect the expression of genes associated with the CpG islands. Typically such hypermethylated genes have reduced or abolished expression, primarily due to down-regulated transcription. Hypermethylation of, for example, PR-B or HOXA10 allows one to also diagnose a cellular proliferative disorder of endometriosis. Using a recently developed PCR-based technique called methylated specific PCR (MSP), aberrantly methylated nucleic acids in biological samples from individuals with endometriosis can be identified.

It is envisioned that such a simple diagnostic test will help clinicians determine whether a patient would respond to conventional progestin treatment for endometriosis, so that they can select an proper treatment modality based on the result of the test, before prescribing any medication to the patient. It is contemplated that at the time of surgery to remove endometriotic lesions, diagnostic assay, such as a hypermethylation assay may be performed on DNA extracted from the lesion to detect PR-B or HOXA10 hypermethylation in a sample. A positive test for hypermethylation of either of these genes allows the clinician to select a patient-specific therapeutic regime to prevent reoccurrence of and treat any existing endometriotic cells. In particular, applicants can detect PR-B hypermethylation in the menstrual blood to predict which patient will respond to progestin treatment.

A suitable embodiment of the invention is a diagnostic test for identifying women with endometriosis who are or may become resistant to progestin/progesterone therapy. A simple test of methylation status using DNA extracted from the endometriotic tissue sample could determine if an individual would respond to progestin/progesterone treatment. Since all drug treatments for treating endometriosis have undesirable, sometimes serious, side effects, this diagnostic test will help clinicians to select a suitable treatment modality from the start of treatment, instead of waiting three to six months after the therapy is ineffective.

In a related embodiment, a method of selecting a therapy for endometriosis is provided. The method includes assaying a biological sample for the presence of endometriotic cells having at least one biomarker associated with endometrial pathology and detecting an hypermethylation of PR-B promoter, aberrant methylation of the HOXA10 gene, or an increase in the expression of DNA methyltransferase in the cells of the endometrial tissue lining the uterus relative to a control cell. Based on the results of the detection assay, a therapy may be selected that includes administering a demethylation agents alone or in combination with histone deacetylase inhibitors (HDACIs).

In a related embodiment, a diagnostic screening method is provided to determine if a subject with endometriosis may be progestin/progesterone resistant. The method includes (a) providing a biological sample (such as menstrual blood) of the subject to be screened; and (b) assaying the biological sample for the presence of a biomarker associated with endometrial pathology, preferably hypermethylation of a progesterone receptor promoter relative to a control, wherein the assay is a DNA screening assay which detects promoter hypermethylation of progesterone receptor (PR-B) or aberrant methylation of the HOXA10 gene. The assay may include but is not limited to methylation analysis, preferably methylation specific PCR, bisulfite sequencing, and other methylation measuring techniques.

In a related embodiment, a diagnostic screening method is provided to determine if a subject with endometriosis may be progestin/progesterone resistant. The method includes (a) providing a biological sample of the subject to be screened; and (b) assaying the biological sample for the presence of a biomarker associated with endometrial pathology, suitably an increase in DNA methyltransferase (DNMT) expression relative to a control. The assay is a RNA abundance measuring assay or an immunoassay, which detects increased expression of DNMT by at least one assay method. Such assays include but is not limited to quantitative RT-PCR of mRNA expression, microarray, and immunofluorescence staining among other suitable assays known in the art.

It is contemplated that the screening tools described herein are capable of being miniaturized and adapted for high throughput screening (HTS).

In a related embodiment, a method is provided for diagnosing endometriosis, including detecting endometriotic cells with a biomarker associated with endometrial pathology. The biomarker is capable of detecting an increased level of DNA methyltransferase (DNMT) expression (DNMT1, DNMT3A and DNMT3), a decreased progesterone receptor-B (PR-B) expression or a hypermethylated PR-B promoter relative to a control sample. Another suitable biomarker is one that is capable of detecting aberrant methylation of HOXA10 gene.

In a related embodiment, a method is disclosed for gauging a subject's predisposition to endometriosis. This is done by taking menstrual blood sample or endometrial cells from the subject; determining the expression pattern of at least one gene selected from the group consisting of PR-B, HOXA10, and the gene coding for DNA methyltransferase in the cells associated with endometrial pathology to determine if the subject has an altered expression pattern in the gene; and diagnosing the subject as predisposed to endometriosis if the expression pattern of the selected gene is altered in the subject as compared to a cell from a normal, non-endometriotic subject.

As used herein, “predisposition” refers to an increased likely that an individual will have a endometriosis, particularly one that is resistant to progesterone therapy. Although a subject with a predisposition does not yet have the disorder, there exists an increased propensity to the disease.

In a related embodiment, a method for monitoring disease progression and/or treatment efficacy and/or relapse of endometriosis is disclosed. The method includes assaying endometrial tissue from the subject for the presence of endometriotic cells having a biomarker associated with endometrial pathology, wherein the biomarker is an increased level of DNA methyltransferase (DNMT) expression (DNMT1, DNMT3A and DNMT3B), a hypermethylated PR-B promoter or an aberrant methylation of HOXA10 gene relative to a control sample.

In a related embodiment, a biomarker associated with and/or predictive of endometriosis is disclosed. The biomarker is used to determine if an endometriosis patient would respond to progestin/progesterone therapy. The biomarker includes an increased expression level of a DNA methyltransferase (DNMT), preferably DNMT1, DNMT3A, and DNMT3B; aberrant methylation of the HOXA10 gene, or hypermethylated progesterone receptor promoter, preferably PR-B isoform; or decreased expression level of a progesterone receptor in a subject with endometriosis relative to a control.

In one embodiment, a method is disclosed for downregulating the expression and/or inhibiting the activity of DNA methyltransferases (DNMT), suitably DNMT1, DNMT3A, and DNMT3B. This can be done administering to a subject in need thereof one or more therapeutic compounds that downregulate DNMT gene expression and/or gene or gene product activity. In a preferred embodiment, a downregulator of the DNMT gene or gene product activity is administered to a human subject for therapeutic treatment (e.g., to ameliorate symptoms or to retard onset or progression) of endometriosis.

In a related embodiment, a method is provided for downregulating the expression of DNA methyltransferases (DNMT) in a subject having endometriosis. The method includes administering an effective amount of a histone deacetylase inhibitor (HDACI) alone or in combination with a demethylation agent (preferably 5-azacytidine) or retinoic acid to the subject. Suitable HDACIs include valproic acid or trichostatin A, TSA. The downregulation of DNMT results in decreased Progesterone receptor (PR) gene methylation, increased PR gene expression, increased progesterone binding, and increased responsiveness to progesterone therapy.

Also provided are methods for preventing or ameliorating symptoms of endometriosis by administering a therapeutic agent. Such agents include but are not limited to: chemical agents that (1) downregulate expression, formation or activity of DNA methyltransferases (DNMT); (2) enhance the expression of the progesterone receptor; or (3) HOXA10 gene; modulate the activity of DNMT, act as agonists of DNMT. Such agonists may include small molecule agonists.

In accordance with this embodiment, the HDACI, preferably valproic acid is administered alone or co-administered with a demethylation agent, (5-azacytidine) or retinoic acid (e.g. orally, intravenously, intramuscularly, subcutaneously, intrathecally, rectally) to a subject. The effect of these compounds on the expression or activity of the DNA methyltransferase (DNMT) is determined. Alterations in the expression and/or activity of DNMT can be assessed by any suitable method described here or known in the art.

In another embodiment, a method is provided for regulating cell proliferation of endometriotic cells. The method includes administering to a subject with endometriosis an effective amount of HDACIs alone or in combination with retinoic acid.

In another embodiment, the invention provides a method of treating endometriosis using an effective amount of HDACIs to reduce the size or completely eliminate endometriotic lesions by inhibiting cell proliferation and more importantly, correction of gene dysregulation, which existing medications can not achieve. Furthermore, it is contemplated that HDACIs have a much better long-term suppression effect than selective progesterone receptor modulators or SPRMS, which have been shown in animal studies to be effective in suppressing proliferation of endometriotic cells in vivo.

In another embodiment, a method is provided for treating a subject having endometriosis by administering to the subject a therapeutically effective amount of HDACIs alone or in combination with a demethylation agent. A suitable demethylation agent is 5-azacytidine and suitable HDACIs include valproic acid (VPA) (Abbott Laboratory). It is believed that the combination may have a synergistic effect in suppressing growth and inflammation of endometriotic cells.

In another embodiment, a method is disclosed for treating subjects with progestin/progesterone-resistant endometriosis by administering an effective amount of an HDACI, suitably valporic acid.

In another embodiment, a method is provided for treating a subject having endometriosis by administering to the subject a therapeutically effective amount of retinoic acid or an HDACI to treat the endometriosis without the side effects associated with conventional progestin/progesterone therapy.

Accordingly, applicants have extensive in vitro evidence to show that HDACIs, administrated alone or in combination with demethylation agents or retinoic acids, can yield desirable effects in endometriotic cells and rectify epigenetic aberrations, thus restoring normal gene expression and rectifying pathological conditions. Also, as described below, applicants have clinical evidence showing that HDACIs function as intended to treat endometriosis and ameleiorate symptoms thereof.

In another embodiment, a kit is disclosed to assay for the presence of promoter hypermethylation of Progesterone Receptor Isoform B (PR-B) in a biological sample relative to a control. A suitable kit includes at least one primer pair or one oligonucleotide capable of selectively hybridizing to the PR-B gene. Also, kits having at least one primer pair or one oligonucleotide for hybridizing to HOXA10 gene are disclosed.

Primers hybridize with target polynucleotide sequences in the PR-B or HOXA10 genes. Nucleic acid sequences including exemplary primers are set forth herein.

Primers of the invention are designed to be “substantially” complementary to each strand of the oligonucleotide to be amplified and include the appropriate G or C nucleotides as discussed above. This means that the primers must be sufficiently complementary to hybridize with their respective strands under conditions which allow the agent for polymerization to perform. In other words, the primers should have sufficient complementarity with a 5′ and 3′ oligonucleotide to hybridize therewith and permit amplification of CpG containing nucleic acid sequence.

The kits can include a carrier means compartmentalized to receive a sample therein, one or more containers comprising a first container containing a reagent which modifies unmethylated cytosine and a second container containing primers for amplification of a CpG-containing nucleic acid, wherein the primers distinguish between modified methylated and nonmethylated nucleic acid. Primers contemplated for use in accordance with the invention include those set forth herein.

In another embodiment, a kit is disclosed to assay for the presence of an increased DNA methyltransferase expression level in a biological sample relative to a control. A suitable kit includes at least one primer pair or one oligonucleotide capable of selectively hybridizing to the DNA methyltransferase (DNMT) gene, where in the DNMTs include DNMT1, DNMT3A, and DNMT3B. The increase in DNMT expression is predictive of endometriosis, suitably the type that is resistant to progestin/progesterone therapy.

In another embodiment, a kit is provided for detecting an increase in the expression level of DNA methyltransferase (DNMT), preferably DNMT1, DNMT3A, and DNMT3B using an antibody. The detection is performed by an immunoassay, such as an ELISA—based assay.

The diagnostic assay protocols described herein are designed to identify promoter hypermethylation of the PR-B gene aberrant methylation of HOXA10 gene, and upregulated expression levels of DNA methyltransferases in a specific, fast and convenient manner. As described herein the assay protocols may be packaged in a kit format. In addition, the disclosed assays may be scaled to accommodate a high through-put format. Thus, the disclosed methods are efficient and readily amenable to high-throughput drug screening protocols. Suitably, the subject assays identify individuals that are susceptible or are resistant to conventional progestin/progesterone therapies for endometriosis.

Furthermore, it is intended that disclosed kits include “Instructions for use,” for how to carry out the described assay protocols (immunoassays or nucleic acid assays). The amounts of the various reagents in the kits can be varied depending on a number of factors, such as the optimum sensitivity of the assay. The instructions for use are suitable to enable an analyst of ordinary skill to carry out the desired assay.

Definitions

For convenience, certain terms employed in the specification, examples, and appended claims are provided here.

As used herein the term “biological sample” refers to any body fluid or tissue. Preferred body fluids include blood, plasma, serum, urine, saliva, sputum, cerebrospinal fluid, mucus, and vaginal and rectal secretions; preferably the biological sample includes blood (suitably menstrual blood) or blood products such as plasma and serum prepared from a blood sample.

As the invention is directed toward the analysis of endometrial pathology, endometrial tissue or cells thereof is a preferred tissue sample and menstrual fluid is most suitable for noninvasively detecting epigenetic aberrations for diagnostic purposes. However the method can be used to analyze other female reproductive tissue as well including tissue from the uterus, cervix, vagina and the like. When tissue samples are used, such as biopsies, they can be homogenized, for example in phosphate buffered saline or, alternatively, in a detergent-containing buffer to solubilize the polypeptides to be detected. It should be noted that although the invention is described primarily with respect to endometrial pathology in humans, it is equally application to all mammalian subjects and, in that regard, has application in veterinary as well as human medical contexts.

As used herein the term “endometriotic cell” refers to cells from endometrial glands and stroma outside the uterine lining. Endometriotic cells can be obtained from endometriotic tissue or menstrual fluid. Endometriotic tissue samples can be obtained from subjects having unexplained infertility, known endometriosis or lower abdominal pain.

As used herein the term “endometrial cell” refers to a cell in the uterine lining.

The term “detect”, “detecting” or “detection” as used herein means determination that a substance, e.g., a biomarker (hypermethylated PR-B, HOXA10 or elevated level of DNMTs) is present. The methods and compositions of this invention can also be used to determine the amount of or concentration of a substance, e.g., biomarker, in a sample. Quantification and detection of biomarkers can be performed by any means known to those skilled in the art. Means of detection and quantification include but are not limited to DNA- or protein based assays such as bisulfite sequencing, and methylation specific PCR or enzyme-linked immuno-sorbant assay (ELISA), respectively.

As used herein the term “diagnosing” refers to diagnosis, prognosis, monitoring, characterizing, selecting patients, including participants in clinical trials, and identifying patients at risk for or having a particular disorder or clinical event or those most likely to respond to a particular therapeutic treatment, or for assessing or monitoring a patient's response to a particular therapeutic treatment.

As used herein the term “biomarker” refers to the hypermethylation of the PR-B gene promoter which effectively silences the progesterone receptor gene is an indicator of ectopic endometriosis. Also, the aberrant methylation (e.g., hypermethylation) of the HOXA10 gene promoter may be responsible for the aberrant gene expression in the endometrium of women with endometriosis. HOXA10 gene aberrant methylation and expression is an indicator of women with endometriosis. Also, an elevated level of DNA methyltrasnferase expression is a marker for endometriosis. These biomarkers for endometriosis are also indicators of resistance to conventional progestin and progesterone therapies. DNA biomarkers, PR-B and HOXA10 can be detected from but is not limited to the menstrual blood, which constitutes a low-cost, potentially high-throughput, and, above all, non-invasive procedure.

As referred to here, a “control” is a normal, healthy cell tissue from a female subject without endometriosis or symptoms thereof. The control is negative for the biomarkers described herein.

As used herein the term “DNA methylation” refers to the covalent modification of cytosine to 5′-methyl cytosine in the genome. In vertebrates, methylation occurs in the context of cytosines followed by guanine, or the so-called CpG sites. DNA methylation in humans is a crucial epigenetic modification of the genome that plays an important role in the regulation of gene expression and genomic imprinting, cell differentiation, and in regulating many cellular processes.

Since, the invention method includes determining the state of methylation of one or more nucleic acids isolated from the subject. The phrases “nucleic acid” or “nucleic acid sequence” as used herein refer to an oligonucleotide, nucleotide, polynucleotide, or to a fragment of any of these, to DNA or RNA of genomic or synthetic origin which may be single-stranded or double-stranded and may represent a sense or antisense strand, peptide nucleic acid (PNA), or to any DNA-like or RNA-like material, natural or synthetic in origin. As will be understood by those of skill in the art, when the nucleic acid is RNA, the deoxynucleotides A, G, C, and T are replaced by ribonucleotides A, G, C, and U, respectively.

A nucleic acid includes a gene that can contain a regulatory region which is a region of DNA that encodes information that directs or controls transcription of the nucleic acid. Regulatory regions include at least one promoter. A “promoter” is a minimal sequence sufficient to direct transcription, to render promoter-dependent gene expression controllable for cell-type specific, tissue-specific, or inducible by external signals or agents. Promoters may be located in the 5′ or 3′ regions of the gene. Promoter regions, in whole or in part, of a number of nucleic acids can be examined for sites of CG-island methylation.

Nucleic acids isolated from a subject are obtained in a biological specimen from the subject. The nucleic acid may be isolated from menstrual blood.

A method for determining the methylation state of nucleic acids is described in U.S. Pat. No. 6,017,704 which is incorporated herein in its entirety and described briefly herein. Determining the methylation state of the nucleic acid includes amplifying the nucleic acid by means of oligonucleotide primers that distinguishes between methylated and unmethylated nucleic acids.

Two or more markers can also be multiplexed in a single amplification reaction to generate a low cost, reliable endometriosis screening test. A combination of DNA markers for CpG-rich regions of nucleic acid may be amplified in a single amplification reaction. The markers are multiplexed in a single amplification reaction, for example, by combining primers for more than one locus. Especially useful are at least two markers including PR-B and HOXA10. The reaction products are separated on a denaturing polyacrylamide gel, for example, and then exposed to film or stained with ethidium bromide for visualization and analysis. By analyzing a panel of markers, there is a greater probability of producing a more useful methylation profile for a subject.

The term “demethylation agent” as used herein refers to a class of compounds capable of removing methylation or suppressing the expression of DNMT1, DNMT3A or DNMT3B. Suitable agents may include 5-azacytidine and derivatives or analogs thereof.

The term “histone deacetylase inhibitors” or “HDACIs” broadly refers to a class of compounds associated with histone deacetyase inhibition, including the short-chain fatty acids (e.g., Butyrate and phenylbutyrate and Valproate), the hyroxamic acids (e.g., the trichostatins, SAHA and its derivatives, Oxamflatin, ABHA, Scriptaid, Pyroxamide, Propenamides), the epoxyketones (e.g., trapoxins, HC-toxin, Chlamydocin, Diheteropeptin, WF-3161, Cyl-1 and Cyl-2), the non-epoxyketone-containing cyclic tetrapeptides (e.g., FR901228, Apicidin, the cyclic-hydroxamic-acid-containing peptides (CHAPs)) the benzamides (e.g., MS-275, CI-994, and other benzamide analogs) and a variety of other miscellaneous chemical structure families (e.g., Depudecin and Organosulfur compounds). Preferred HDACIs for treating endometriosis include trichostatin A, TSA or valproic acid, VPA and derivatives or analogs thereof.

Related benefits of using HDACIs, suitably valproic acid, alone or in combination with other compounds such as retinoic acid, or demethylation agents for treating endometriosis, include suppression of proliferative endometriotic cells, restoration of aberrant gene expression, reduces the need to use hormonal therapy. One HDACIs, valproic acid, is also FDA-approved for treating diseases such as seizures, bipolar disorders, and cancer, so it is known to exhibit favorable pharmacokinetic properties. Other novel HDACIs, presumably with higher specificity and potency with less side effects, are also emerging. The parameters for assessing successful treatment and improvement in the disease are readily measurable by routine procedures familiar to a physician.

In another embodiment, it is contemplated that in treating endometriosis HDACIs, preferably valproic acid may be co-administered either sequentially or simultaneously with demethylation agents, preferably, 5-azacytidine or retinoic acid (RA). It is believed that these combinations of demethylation agents and HDACIs have a synergistic effect in suppression of growth and inflammation.

Interestingly, we discovered that use of 5-azacytidine or retinoic acid alone has some effect in suppression of growth (but not inflammation). This effect may be a result of the multiple pathways involved in the pathogenesis of endometriosis. It is believed that the joint use of RA and HDACIs suppresses more than one pathway resulting in the observed synergistic effects.

It is contemplated that endometriotic cells may be treated with HDACI and retanoic acid (including for example 9-cis RA and all-trans RA) has a synergistic effect of inducing cell cycle arrest such that the cell becomes dormant.

As used herein, the term “immunoassay” refers to a laboratory test based on an “antigen-antibody” reaction. The presence of specific antigens and antibodies in the body can be a very clear indication of specific diseases or functional disorders. The measurement of an antigen-antibody interaction is accomplished utilizing such procedures as immunofluorescence, radioimmunoassay, enzyme immunoassay or other nonradioisotopic techniques. In accordance with the invention, immunoassays include enzyme linked immunoabsorbent assay (ELISA), fluorescent immunosorbent assay (FIA), chemical linked immunosorbent assay (CLIA), radioimmuno assay (RIA), and immunoblotting. For a review of the different immunoassays which may be used, see: The Immunoassay Handbook, David Wild, ed., Stockton Press, New York, 1994.

In one embodiment, the invention provides a method for diagnosing endometriosis, preferably progestin/progesterone resistant endometriosis in a subject by employing for example, an immunoassay to detect an increased expression of DNA methyltransferases, preferably DNMT1, DNMT3A and DNMT3B relative to a control sample. Preferred antibodies used in detecting increased DNA methyltransferase expression in subject with endometriosis include primary antibodies to DNMT1, DNMT3A and DNMT3B.

In a typical assay, the reagents include a serum sample from a subject, the antibodies to be detected (contained in the serum sample), antigen, and means for producing a detectable signal.

The signal producing system is made up of one or more components, at least one of which is a label, which generate a detectable signal that relates to the amount of bound and/or unbound label i.e., the amount of label bound or unbound to the antigen. The label is a molecule that produces or which may be induced to produce a signal. Examples of labels include fluorescers, enzymes, chemiluminescers, photosensitizers or suspendable particles. The signal is detected and may be measured by detecting enzyme activity, luminescence or light absorbance. Radiolabels may also be used and levels of radioactivity detected and measured using a scintillation counter.

Examples of enzymes which may be used to label the anti-human immunoglobulin include beta-D-galactosidase, horseradish peroxidase, alkaline phosphatase, and glucose-6-phosphate dehydrogenase (“G6PDH”). Examples of fluorescers which may be used to label the anti-human immunoglobulin include fluorescein, isothiocyanate, rhodamine compounds, phycoerythrin, phycocyanin, allophycocyanin, o-phthaldehyde and fluorescamine. Chemiluminescers include e.g., isoluminol.

As used herein, the term “antibody” includes intact molecules as well as fragments thereof, such as Fab and F(ab′)2, which are capable of binding the epitopic determinant. Antibodies that bind the genes or gene products of the present invention can be prepared using intact polynucleotides or polypeptides or fragments containing small peptides of interest as the immunizing antigen attached to a carrier molecule.

For the production of antibodies which may be either monoclonal or polyclonal, an immunogen (described above) is administered to an animal (route of administration may be via a number of ways such as subcutaneous, intradermal, intraperitoneal, etc.) according to standard protocols. Preferably, the animal is a New Zealand White Rabbit, but one may use other animals (e.g., mice, sheep, pigs, goats, donkeys). It is preferable that the immunogen is emulsified using an adjuvant, however, the use of adjuvants is not necessarily required. Preferably, following at least two immunizations, serum is obtained from the animal and tested for specificity according to standard procedures (e.g., screen using solid phase ELISA techniques). The antiserum obtained in this case is polyclonal. This antiserum may be used as crude antiserum or may be further purified using standard procedures.

As used herein, the term “level” refers to expression levels of RNA and/or protein or to DNA copy number of a marker of the present invention. Typically the level of the marker in a biological sample obtained from the subject is different (i.e., increased or decreased) from the level of the same variant in a similar sample obtained from a healthy individual.

“Treatment” refers to therapy, prevention and prophylaxis and particularly refers to the administration of medicine or the performance of medical procedures with respect to a patient, for either prophylaxis (prevention) or to cure or reduce the extent of or likelihood of occurrence of the infirmity or malady or condition or event in the instance where the patient is afflicted, preferably progestin/progesterone-resistant endometriosis. Specifically, treatment refers to administering orally, parenterally, or intravenously either alone or in combination with other therapeutic agents, so that the endometriotic cell proliferation is inhibited, adhesion formation is reduced and the pain is relieved.

It is envisioned that successful treatment would (1) relieve/reduced pain and inflammation symptoms, (2) shrink or slow endometrial growths and lesions, (3) preserve or restore fertility, and (4) prevent/delay recurrence of the disease. Those in need of treatment include those already with the disorder as well as those prone to have the disorder or those in whom the disorder is to be prevented. It is envisioned that gene dysregulation may also be corrected through such a treatment regime.

In a preferred embodiment the compounds described herein are orally administered to a subject, preferably a human subject. It is contemplated that an oral formulation of HDACIs will provide a good safety profile, with attainment of targeted therapeutic serum drug levels, and pharmacokinetics that support twice a day or once a day dosing.

An “effective amount” or “therapeutically effective amount” may be determined empirically and in a routine manner, in relation to the stated purpose. More specifically, the term “therapeutically effective amount” refers to an amount of a compound effective to “treat” a disease or disorder in a subject. In the case of endometriosis, the therapeutically effective amount of the compounds may reduce the number of affected cells; cell size and/or number of endometriotic cells; inhibit (i.e., slow to some extent and preferably stop) endometriotic cell infiltration into peripheral organs (i.e., invasion); cause endometrial cell cycle arrest and/or relieve to some extent one or more of the symptoms associated with the endometriosis. Also, it is envisioned that an effective amount of the HDACI's alone or in combination with demathylation agents and retinoic acid will be cytotoxic to unwanted endometriotic cells (i.e. ectopic endometrial cells) but cause no harm to endometrial cells (eutopic endometrial cells) since endometrial cells are part of the functional organ, the uterus.

Notably, in regards to HDACIs, demethylation agents and retinoic acids disclosed herein, those skilled in the art would be able to readily ascertatin the dosage level required for therapeutic efficacy through routine optimization.

As used herein the term “subject” refers to any mammal, including humans, human fetus in utero, domestic and farm animals, and zoo, sports, or pet animals, such as dogs, cats, cattle, horses, sheep, pigs, goats, rabbits, etc. Suitably, the mammal is human with endometriosis or a carrier of the epigenetic disease.

In another embodiment, it is contemplated that the compounds disclosed here are administered in a pharmaceutical composition containing a pharmaceutically acceptable carrier. Such as carrier includes pharmaceutically acceptable carriers, excipients, or stabilizers which are nontoxic to the cell or mammal being exposed thereto at the dosages and concentrations employed. A carrier may be liquid or solid and suitable for oral administration. Examples of physiologically acceptable carriers include buffers; antioxidants; low molecular weight polypeptide; proteins; hydrophilic polymers; amino acids; carbohydrates; chelating agents; sugar alcohols; salt-forming counterions; and/or nonionic surfactants.

In a suitable embodiment, the pharmaceutical composition may be in the form of a tablet or capsule. The pharmaceutical compositions may include an additional active agent. It is also envisioned that the pharmaceutical composition may be co-administered with other compounds for the treatment endometriosis described herein.

As used herein, the term “administration” means alone or “in combination with” one or more further therapeutic agents includes simultaneous (concurrent) and consecutive administration in any order. In addition to oral administration, it is contemplated that the compound may be administered to humans for therapy by other suitable routes of administration, including nasally, as by, for example, a spray, rectally, intravaginally, parenterally (intravenous, subcutaneous, or intramuscular injection), intracistemally and topically, as by powders, ointments or drops, including buccally and sublingually.

Regardless of the route of administration selected, the compounds of the present invention may be used in a suitable hydrated form, and/or pharmaceutical composition formulated into pharmaceutically-acceptable dosage forms, such as described below or by other conventional methods known to those of skill in the art.

The selected and actual dosage levels of the pharmaceutical compositions of this invention can be varied to obtain an amount of the active ingredient effective to achieve the desired therapeutic response for a particular individual, composition, and mode of administration, without being toxic to the patient.

Accordingly, it is envisioned that the dosage levels will vary depending upon a variety of factors. Such factors include the activity of the compound(s) employed, or the ester, salt or amide thereof, the route of administration, the time of administration, the rate of excretion of the particular compound being employed, the duration of the treatment, other drugs, compounds and/or materials used in combination with the particular apoptosis-inducing agent employed, the age, sex, weight, condition, general health and prior medical history of the patient being treated, and like factors well known in the medical arts. A physician or veterinarian having ordinary skill in the art can readily determine and prescribe the effective amount of the pharmaceutical composition required.

In general, physicians skilled in the art would readily be able to prescribe a suitable daily dose of the HDACIs and demethylation agents described herein that will be effective to produce a therapeutic effect to treat endometriosis. Such an effective dose will depend upon the factors described above.

In one embodiment, a method is provided for inhibiting proliferation of endometriotic cells by administering to a cell sample a therapeutically effective amount of HDACIs, preferably valproic acid alone or in combination with retinoic acid. It is contemplated that the upregulation of PR-B and AR may be responsible for antiproliferative effects induced by TSA, a histone deacetylase inhibitor (HDACI). In accordance with the invention, HDACIs may be a promising therapeutic in treating endometriosis due to their antiproliferative effects as well as the potential to restore gene dysregulation through chromatin remodeling.

It is to be understood that this invention is not limited to the particular methodology, protocols, cell lines, animal species, and reagents described, as such may vary. It is also to be understood that the terminology used herein is for the purpose of describing particular embodiments only, and is not intended to limit the scope of the present invention which will be limited only by the appended claims.

### EXAMPLES

### Example 1

### Aberrant Methylations in Endometriosis

The physiological effects of progesterone (P) is mediated by two isoforms of progesterone receptors (PRs): PR-A and PR-B (as defined by Gene ID: 5241). Progestins have long been used in the treatment of endometriosis but unfortunately the relief of pain is relatively short-term. In addition, about 9% of women with endometriosis simply do not respond to progestin therapy due to reasons unknown. In fact, a general tendency for relative progesterone resistance within eutopic and ectopic endometrium of women with endometriosis and the downregulation of PR-B, but not PR-A, in endometriosis have been noted. Since promoter hypermethylation is well-documented to be associated with transcriptional silencing, we sought to determine the methylation status of the PR-A and PR-B promoter regions in the epithelial component of endometriotic implants using a combination of laser capture microdissection (LCM), methylation specific PCR, and bisulfite sequencing. We found that the promoter region of PR-B, but not PR-A, is hypermethylated in endometriosis as compared with controls. In addition, the PR-B expression was significantly reduced in the ectopic endometrium. Our finding suggests that progesterone resistance in endometriosis in general and the downregulation of PR-B, but not PR-A, in particular, are a result of promoter hypermethylation of PR-B, but not PR-A. This, in conjunction with our reported aberrant methylation of HOXA10 in the eutopic endometrium of women with endometriosis (4), strongly suggests that endometriosis is an epigenetic disease. Since there are about 10% of women with endometriosis who do not respond well to progestin treatment, it is plausible that those with hypermethylated PR-B promoter may have reduced or no PR-B expression, rendering them unresponsive to progestin therapy. Thus, it is contemplated that endometriosis, particularly progesterone/progestin resistant endometriosis may be effectively treated through reversing aberrant methylation via pharmacological means.

Tissue Collection and Laser Capture Microdissection (LCM)

Endometriotic and endometrial tissues were taken from 12 patients (cases), aged 18 to 36 years, with surgically and histologically confirmed rAFS stages II-IV endometriosis. Among these 12 patients, 8 ectopic endometrial tissue samples and 6 eutopic endometrial tissue samples were available for analysis. Control samples of endometrial biopsies were obtained from 4 healthy women, aged 26-38 years, undergoing tubal sterilization and laparoscopically confirmed to be free of endometriosis. Since the DNA methylation status is unlikely to be affected by the menstrual phase, the information on the phase of menstrual cycle at the time of tissue harvesting was determined according to the criteria of Noyes et al. (Fertil Steril, 1950, Vol. 1 pg. 3-25). All endometriotic and endometrial tissue samples were snap frozen on dry ice immediately after surgical dissection and then stored in a −80° C. freezer.

The tissue section slides were stained with HistoGene section reagents (Arcturus, Mountain View, Calif.). To minimize contamination from other cell types, epithelial cells were harvested from eutopic and ectopic endometrial tissue samples, using LCM through a Pixcell II laser capture microscope system (Arcturus) as described previously (Wu et al. Fertil Steril, 2003, 79 Suppl. 1:p. 710-7). Approximately 3,000 captured epithelial cells were then incubated at 37° C. overnight with 50 μl of proteinase K lysis buffer (1 mg/ml proteinase K+1% Tween-20 in 1×TE, pH8.0), Sigma, St. Louis, Mo.] for DNA extraction. Approximately 5 ng of genomic DNA was isolated from each cell lysate using the standard Phenol/Chloroform extraction method. The DNA from blood samples was extracted using Puregene DNA isolation kit (Gentra, Minneapolis, Minn.). It is noted that this research was approved by the Institutional Review Board of MCW. All participating subjects signed a written informed consent before recruitment to the study.

Methylation Specific PCR (MSP)

All samples were initially screened at PR-A and PR-B regions by MSP as described previously (Herman et al., Proc Natl Acad Sci USA, 1996, 93(18): p. 9821-6). When the CpG sites in the region analyzed by MSP are methylated, the M (methylated) band would show up. On the other hand, the U (unmethylated) band would be present when the sites are unmethylated. Occasionally, both bands could be present if the sites are partially methylated. The primers designed for PR-A and PR-B were described previously and were as follows: 1) PR-A, methylated: forward 5′-ACGGGTTATTTTTTTTTCG-3′ (SEQ ID NO: 1), and reverse 5′-TAAAATATACGCCCTCCACG-3′ (SEQ ID NO: 2); unmethylated: forward 5′-ATGGGTTATTTTTTTTTTG-3′ (SEQ ID NO: 3), and reverse 5′-TAAAATATACACCCTCCACA-3′ (SEQ ID NO: 4). 2) PR-B, methylated: forward: 5′-TGATTGTCGTTCGTAGTACG-3′ (SEQ ID NO: 5), and reverse 5′-CGACAATTTAATAACACGCG-3′ (SEQ ID NO:6); unmethylated, forward 5′-TGATTGTTGTTTGTAGTATG-3′ (SEQ ID NO:7), and reverse 5′-CAACAATTTAATAACACACA-3′ (SEQ ID NO:8).

Bisulfite Sequencing of PR-A and PR-B

The methylation statuses of PR-A and PR-B were further validated by bisulfite sequencing. Genomic DNA extracted from endometrial tissues was modified with bisulfite reagents following the manufacturer's instructions (Zymo Research, CA). This modification resulted in a conversion of unmethylated cytosine to thymine, whereas the methylated cytosine remained unchanged. A total of 20 ng of bisulfite-modified DNA was subjected to PCR amplification and directly sequenced using the ABI 3700 automated sequencing system (Applied Biosystems, CA). The following primers were designed to amplify CpG-rich regions within PR-A and PR-B: (1) PR-A: forward, 5′-GGTTTTGTTAGGGATAGGATTTTTT-3′ (SEQ ID NO: 9), and reverse, 5′-ACTACCTCCAACACCCCTTATAACT-3′ (SEQ ID NO: 10); (2) PR-B: forward 5′-AGTATGGAGTTAGTAGAAGTT-3′ (SEQ ID NO: 11), and reverse 5′-TCACAAGTCCAACACTTAAATAACT-3′ (SEQ ID NO: 12).

Quantitative Real-Time RT-PCR

To measure PR-B mRNA abundance for the available tissue samples; O.C.T (Sakura Finetek, Torrance, Calif.) embedded-frozen endometrium tissue sections were stained using Histogene LCM Frozen Section Staining solutions (Arcturus, Mountain View, Calif.). The glandular epithelial cells were dissected by LCM. Total RNA was extracted from the captured epithelial cells using the Picopure RNA isolation kit (Arcturs) and treated with RNase-free DNase I (Invitrogen). The total RNA (10 ng) was then reverse-transcribed (Superscript II Reverse Transcriptase, Invitrogen) and the expression levels of PR-B were quantified by a real-time RT-PCR analysis using SYBR Green I detection kit (Qiagen). PCR reactions were carried out on the ABI PRISM 7900HT Sequence Detection System. The amount of mRNA level of PR-B from each subject was normalized with that of 18s mRNA (endogenous control, Ambion) taken from the same subject and the relative expression level of PR-B was calculated using relative quantification of gene expression (Applied Biosystems). The primers selected for RT-PCR yielded 442 bp PCR product from PR-B mRNA. The PR-B cDNA primers were: forward 5′-TAGTGAGGGGGCAGTGGAAC-3′ (SEQ ID NO:13), reverse 5′-AGGAGGGGGTTTCGGGAATA-3 (SEQ ID NO: 14).

Data Analysis

For bisulfite sequencing results, the methylation patterns of each subject were listed along CpG sites sequenced for a given region. For each CpG site, no methylation was coded as 0, partial methylation, 0.5, and complete methylation, 1. Partial methylation occurred when equal or nearly equal amounts of C and G were present in the sample, manifested as an identical or nearly identical peak in the DNA sequencer, thus precluding an unambiguous base calling. For each subject, scores across all sequenced CpG sites in the same region were added. The difference in scores between the cases and the controls was compared and evaluated by a randomization test by adding scores in a group (case or control), as previously described (Wu et al., Am J Obstet Gynecol, 2005. 192.).

Briefly, 100,000 permutations were conducted to arrive the empirical p-value,

$$p = \frac{{\sum }_{i} {I}_{i}}{n} ,$$

where n is the number of permutations, and Iᵢ is the indicator variable indicating whether or not the total score Sᵢ in the patient group at the ith permutation exceeds the observed score S₀. For MSP results, data were coded similarly for unmethylation, partial methylation, and complete methylation for each patient and a randomization test was performed to see whether the difference between the groups of interest is significant or not. P<0.05 was considered to be statistically significant. All computations were carried out with R (version 2.2.0, www.r-project.org).

Results

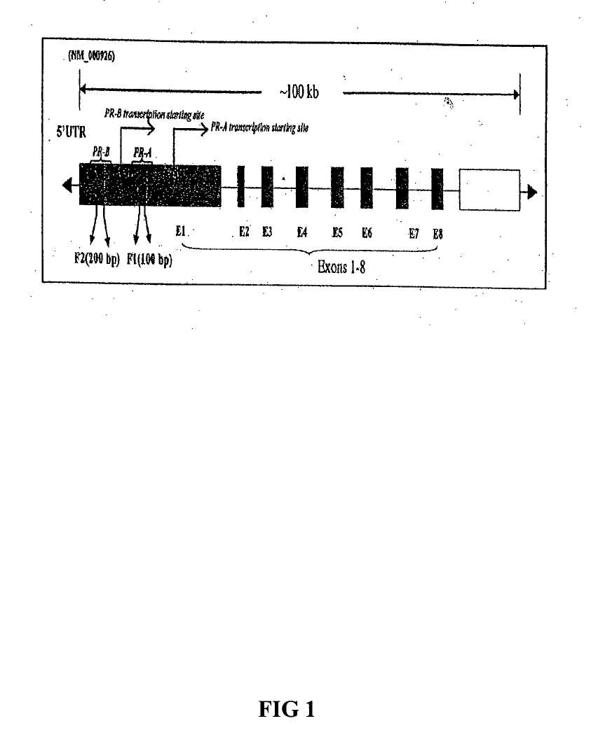

The genomic structure of human PR gene, along with the fragments in the promoter regions of the two isoforms that were sequenced after bisulfite treatment, is depicted in FIG. 1.

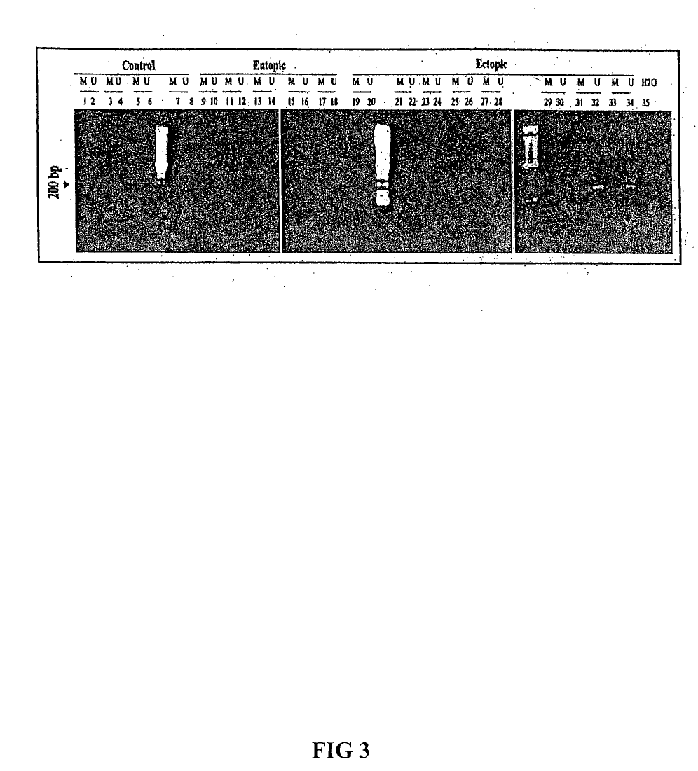

The information on the characteristics of the patients and controls were listed in FIG. 3. The mean age (standard deviation) of subjects in the ectopic endometrium group (case), the eutopic endometrium group (case) and the control group was 30.1 (5.9), 34.5 (1.6), and 31.8 (6.1) years old, respectively. Hence, the ages of the ectopic endometrium (cases) and control groups are comparable, but the patients in the eutopic endometrium group were slightly older.

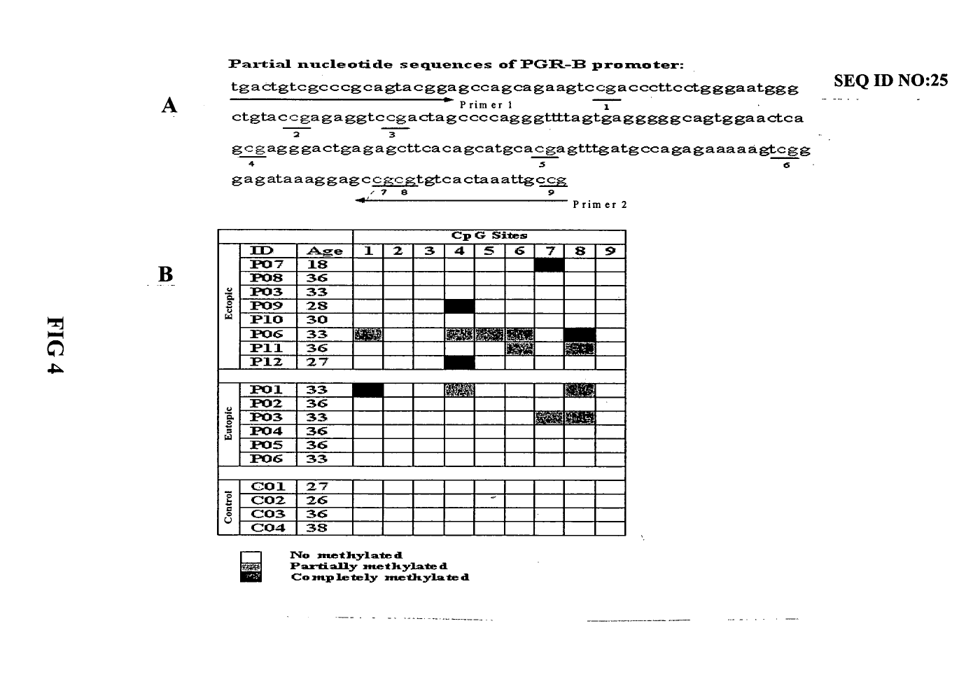

In the MSP assay, all samples yielded only unmethylated bands at PR-A, suggesting that there is no apparent methylation at PR-A (data not shown). At PR-B, in contrast, 6 (lanes 19, 21, 25, 29, 31, and 33) out of 8 endometriotic (ectopic) samples, 2 (lanes 15 and 17) out of 5 eutopic endometrial samples, but none in the control samples, had both methylated and unmethylated bands (FIG. 4), while the other 2 ectopic and 3 eutopic samples as well as all 4 control samples showed only unmethylated bands (FIG. 4). These results suggest that the ectopic endometrial samples have a stronger tendency to be partially methylated in the promoter region of PR-B (p=0.03, two-sided test, based on a randomization test of 20,000 permutations). The difference in the methylation status in the eutopic endometrium between the cases and controls was not statistically significant (p=0.45, two-sided test, based on a randomization test of 20,000 permutations).

The MSP analysis of PR-B was further confirmed by bisulfite sequencing, which is considered to be the gold standard for methylation evaluation (Attia et al., J Clin Endocrinol Metab, 2000. 85(8): p. 2897-902) (FIG. 3). As shown in the FIG. 3, the PR-B promoter region contains 9 CpG sites and all samples were successfully sequenced. In addition, as a group, ectopic implants were hypermethylated in the promoter region of PR-B, as compared with controls (P=0.012, two-sided test, based on 20,000 permutations). Although the eutopic endometrium of women with endometriosis also appeared to be slightly more hypermethylated than the controls, the difference from the control group was marginally significant (P=0.076, two-sided test, based on 20,000 permutations). Among 8 ectopic implants, 3 did not show any methylation at all, suggesting that PR-B promoter hypermethylation may be heterogenous in ectopic implants. This is consistent with the reported heterogenous immunohistochemical staining of PR in ectopic implants (Lessey et al., Fertil Steril, 1989, 51(3): p. 409-15). For the fragment of PR-A (FIG. 1), we did not find any difference in methylation patterns between controls and ectopic implants (data not shown).

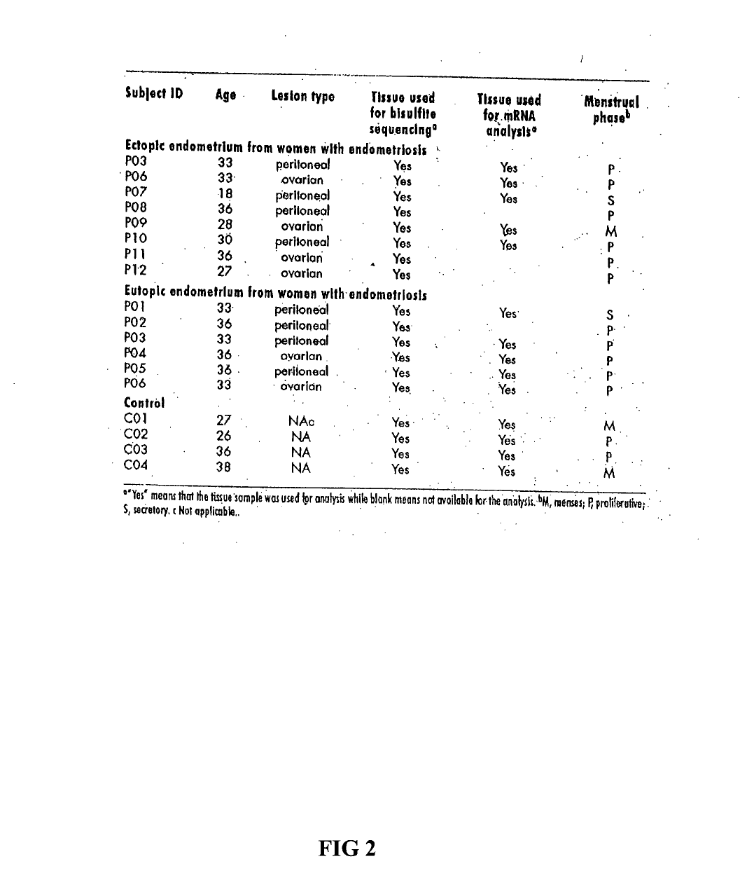

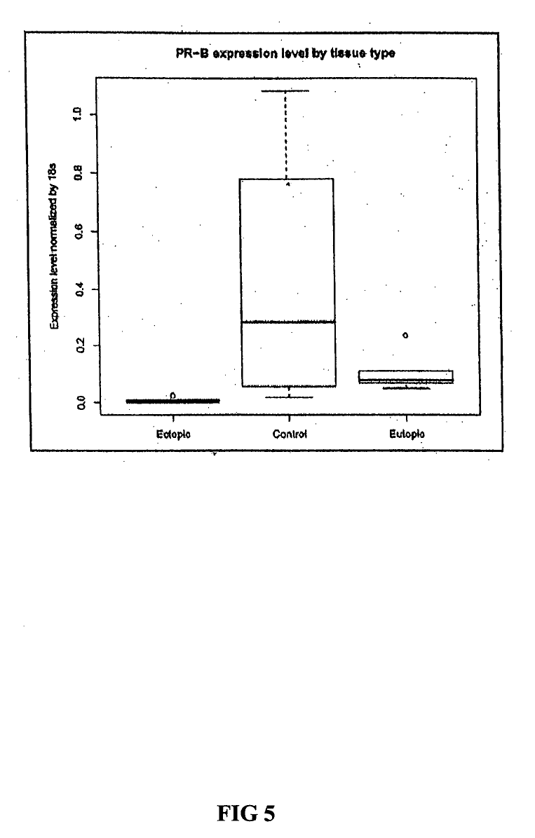

We also performed quantitative RT-PCR to measure PR-B mRNA abundance for the available tissue samples (FIG. 2), using 18s as a normalization control. The results were presented in FIG. 5. It clearly shows that the expression level of PR-B was significantly lower than the control group (p=0.025, two-sided randomization test based on 20,000 permutations), which agrees very well with the previous reports (Attia et al., J Clin Endocrinol Metab, 2000. 85(8): p. 2897-902). Although the PR-B expression level in the eutopic endometrium was apparently lower than the control (FIG. 5), the difference did not reach statistical significance (p=0.12, two-sided test, based on 20,000 permutations). Therefore, the PR-B promoter hypermethylation in ectopic endometrium was associated downregulation of PR-B expression.

### Example 2

### Aberrant Expression of DNA Methyltransferases (DNMTs) in Endometriosis

DNA methylation is catalyzed by DNA methyltransferases (DNMTs). There are four active DNA methyltransferases have been identified in mammals. They are named DNMT1, DNMT2, DNMT3A and DNMT3B. DNMT1 (Gene ID: 1786) is the most abundant DNA methyltransferase and it predominantly methylates hemimethylated CpG di-nucleotides in the mammalian genome. This enzyme is 7-20 fold more active on hemimethylated DNA as compared with unmethylated substrate in vitro, but it is still more active at de novo methylation than other DNMTs. The enzyme is about 1620 amino acids long. The first 1100 amino acids constitute the regulatory domain of the enzyme, and the remaining residues constitute the catalytic domain. These are joined by Gly-Lys repeats. Both domains are required for the catalytic function of DNMT1.

DNMT3 is a family of DNA methyltransferases that could methylate hemimethylated and unmethylated CpG at the same rate. The architecture of DNMT3 enzymes is similar to DNMT1 with regulatory region attached to a catalytic domain. The members of the DNMT3 family that are relevant to this application are DNMT3a and 3b. DNMT3a (Gene ID: 1788) and DNMT3b (Gene ID: 1789) can mediate methylation-independent gene repression. DNMT3a can co-localize with heterochromatin protein (HP1) and methyl-CpG binding protein (MeCBP). They can also interact with DNMT1, which might be a co-operative event during DNA methylation. DNMT3a prefers CpG methylation to CpA, CpT, and CpC methylation, though there appears to be some sequence preference of methylation for DNMT3a and DNMT3b. DNMT3a methylates CpG sites at a rate much slower than DNMT1, but greater than DNMT3b.

To determine that DNMTs were aberrantly expressed in endometriosis, we evaluated the expression levels of DNMT1, DNMT3A and DNMT3B in epithelial cells of ectopic implants. In many cancers where aberrant methylations have been found, these three genes are reported to be over-expressed. Over-expression of these genes are shown to be associated with poorer prognosis in some cancers.

Using LCM, we harvested epithelial cells from eutopic and ectopic endometrium of women with and without endometriosis and measured expression levels of DNMT1, DNMT3A, and DNMT3B for 14 ectopic implants and 8 eutopic endometrial tissue samples from women with laparoscopically diagnosed endometriosis, with 10 eutopic endometrial tissue samples from women free of endometriosis who underwent tubal ligation serving as controls.

Using quantitative real-time RT PCR, we found that the expression levels of all three DNMTs, normalized to the expression level of proliferating cell nuclear antigen (PCNA), are significantly higher in ectopic implants as compared with controls, while in eutopic endometrium of women with endometriosis their expression levels are not significantly different. Normalization to s 18 yielded similar results. This agrees with our finding in gene expression study in that the DNMT1 is differentially expressed in ectopic and eutopic endometrium 173. Since DNMT1 targets replication foci by binding to PCNA, normalization to PCNA expression seems to be appropriate.

Although this analysis did not control for menstrual phase, normalization to PCNA has, at least in part, adjusted for the difference in proliferation during the different phases of the menstrual cycle. This finding further supports the notion that endometriosis may be an epigenetic disease and suggests that aberrant methylation may be more wide spread than we previously thought.

Tissue Collection

After informed consent, endometriotic and endometrial tissues were taken from 17 patients with endometriosis (cases), aged 25 to 51 years, with surgically and histologically confirmed rAFS stages II-IV endometriosis. Among them, 6 patients had both endometrial and endometriotic tissue samples, while the rest only had either endometrial or endometriotic tissue sample. Endometrial biopsies were obtained from 8 healthy women undergoing tubal sterilization, aged 22-42 years, laparoscopically confirmed to be free of endometriosis, and were used as controls. For each subject, her phase of menstrual cycle at the time of tissue harvesting was determined according to the criteria of Noyes et al (Fertil Steril, 1950, Vol. 1 pg. 3-25). All endometriotic and endometrial tissue samples were snap frozen on dry ice immediately after surgical dissection and then stored in a −80° C. freezer. This research was approved by the Institutional Review Board of MCW.

Sample Preparation

Tissue sections were prepared as previously described (Wu et al. 2005 Transcriptional characterizations of differences between eutopic and ectopic endometrium. Endocrinol in press) and incorporated by reference herein its entirety. Approximately 2,000 epithelial cells were harvested from each sample using laser capture microdissection through a Pixcell II laser capture microscope system (Arcturus Engineering, Mountain View, Calif.).

RNA Isolation and cDNa Synthesis

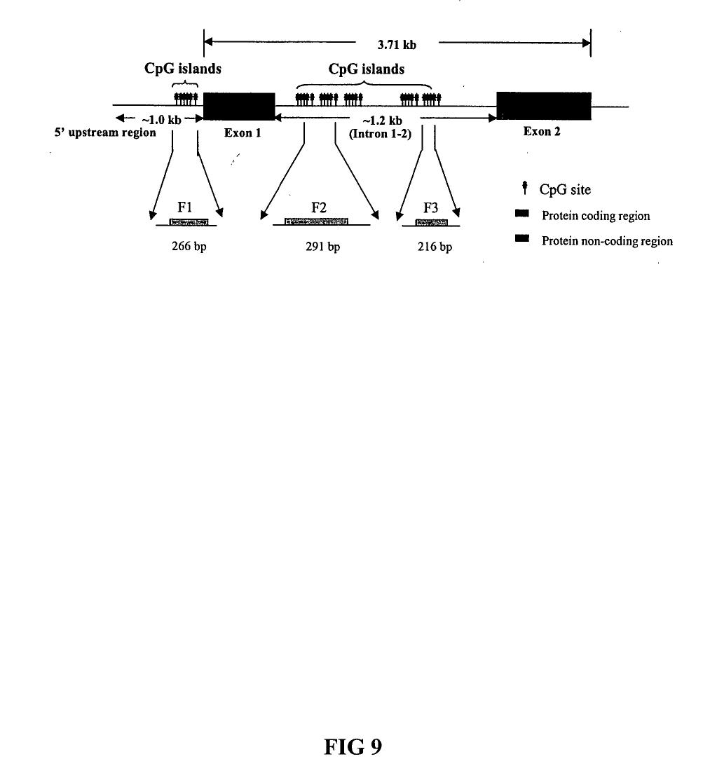

Total RNA was isolated from epithelial cells using a RNA isolation kit from Arcturus. It was treated with DNase I to remove potential DNA contamination and then reverse-transcribed using Superscript II Reverse Transcriptase (Invitrogen, Carlsbad, Calif.). First-strand cDNA obtained from each RNA sample was stored at −80° C. until use. To measure the mRNA abundance of DNMT1, DNMT3A and DNMT3B in each sample, semi-quantitative RT-PCR was initially performed. The mRNA abundance of PCNA and GAPDH were used as proliferative and endogenous controls, respectively. The cDNA primers designed for subsequent PCR were listed in FIG. 9.

Real-Time RT-PCR

Quantitative real-time RT-PCR was carried out on an ABI 7900 (Applied Biosystems), and monitored by SYBR Green I (Qiagen, Valencia, Calif.). The PCR products of the expected size were visualized on a 0.8% agarose gel. The relative mRNA level of each gene was calculated using relative quantitation of gene expression (Applied Biosystems, Foster City, Calif.). The means of three replicated measurements were calculated and shown as mean ±s.d.

Immunofluorescence Staining

Expression of DNMT1 in epithelial cells were detected using rabbit anti-DNMT1 and FITC conjugated goat anti-rabbit IgG (Santa Cruz biotechnology, CA). Mouse monoclonal anti-cytokeratin (8/18) (NCL-5D3, Novcastra, Newcastle upon Tyne, UK) and FITC conjugated goat anti-mouse IgG (Biomeda, Foster city, Calif.) were used to distinguish epithelial cells from stromal cells in glands.

The frozen tissue sections were initially fixed in 100% cold acetone for 2 minutes. After washing with cold PBS, sections were blocked with PBS containing 10% normal goat serum (blocking solution) for 2 h at room temperature. The primary antibody for DNMT1 was diluted at 1:50, the antibody for cytokeratin (8/18) was diluted at 1:25 dilution in blocking solution, and were both incubated on the sections for overnight at 4° C. Sections treated without primary antibody served as negative control. Following washing with cold PBS, FITC conjugated secondary antibody diluted at 1:500 for DNMT1 and 1:200 for cytokeratin (8/18) were added to the section and incubated for 2 hr at room temperature. After washing with PBS, the sections were mounted by cover slips with anti-fade mounting medium (Biomeda, Foster city, Calif.). Fluorescence staining was visualized using Nikon E-600 Epi-Fluorescence Microscope.

Statistical Analysis

For semi-quantitative RT-PCR data, unpaired t-test with unequal variances was used to test whether there is difference in mean between ectopic or eutopic endometrium and the control group, and paired t-test was used for the paired ectopic and eutopic data. Due to skewed distribution of data, a randomization test was employed for quantitative real-time RT-PCR data to test whether the group means are different. All randomization tests were based on 20,000 permutations under the null hypothesis of no difference. Sample median of each group was presented since sample median is more statistically robust than sample means. To test the significance of Pearson's correlation coefficient, the t-test based on Fisher's Z transform was used. All data were square-root transformed before the test. All computations were carried out in R 2.2.0, a language and environment for statistical computing and graphics. The significance level was set at 5%.

Results

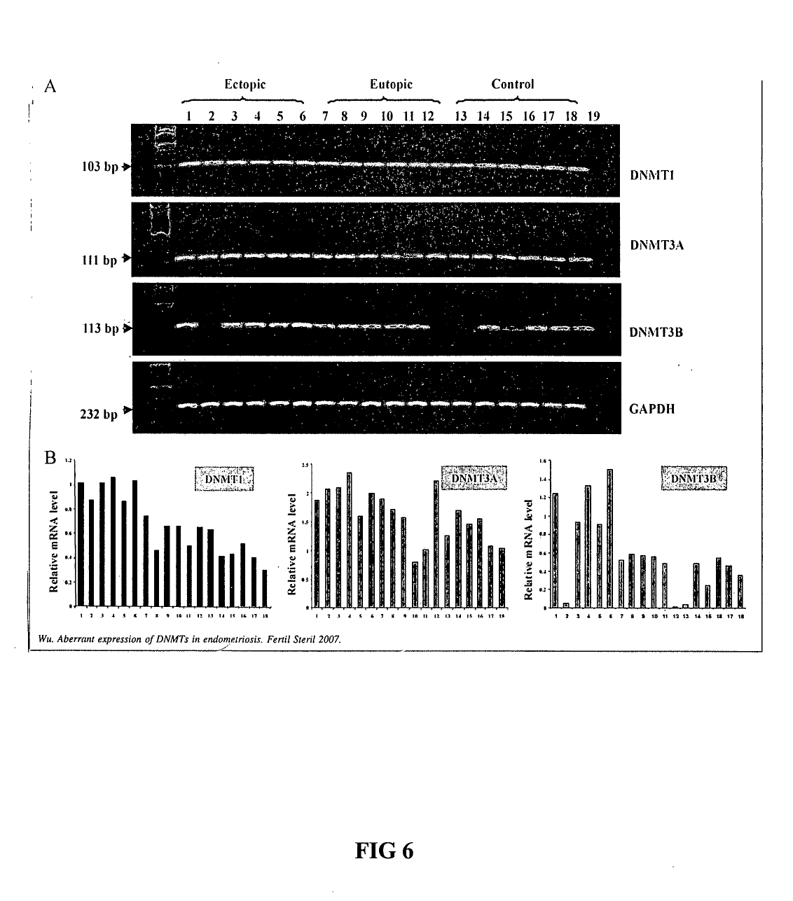

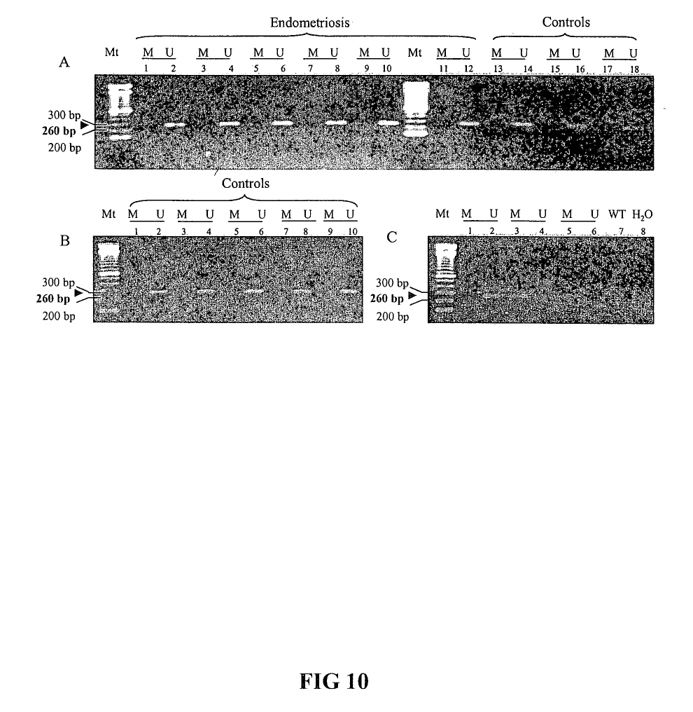

Seventeen women with endometriosis and eight women without endometriosis who underwent tubal ligation were recruited to this study. Their characteristics were listed in FIG. 10. The mean ages of the cases and controls were 35.5 (S.D.=6.4) and 30.5 (S.D.=7.4) years old, respectively. We initially examined the expression levels of DNMT1, DNMT3A and DNMT3B by semi-quantitative RT-PCR analysis. We harvested epithelial cells from 6 pairs of ectopic eutopic endometrial tissue samples using laser capture microdissection. Epithelial cells captured from 6, loosely age-matched, endometriosis-free women who underwent tubal ligation were used for controls. PCR products corresponding to DNMT1 (103 bp), DNMT3A (111 bp), DNMT3B (113 bp), and GAPDH (232 bp) were generated (FIG. 6A). The expression levels of DNMT1, DNMT3A and DNMT3B relative to GAPDH were determined (FIG. 6B). The Expression level of DNMT1 was significantly higher in ectopic endometrium (P<0.0001) and eutopic endometrium (P=0.03) as compared with controls (mean relative expression level ± standard deviation: ectopic, 0.96±0.08; eutopic, 0.60±0.10; control, 0.45±0.45). The expression level of DNMT3A was higher (P=0.001) in ectopic endometrium as compared to its paired counterpart and controls (ectopic, 2.0±0.24; eutopic, 1.54±0.53; control, 1.35±0.27). Similarly, the expression level of DNMT3B was higher (P=0.028) in ectopic endometrium as compared with its paired eutopic endometrium and controls (ectopic, 0.99±0.51; eutopic, 0.45±0.21; control, 0.35±0.18). The expression levels of DNMT3A and DNMT3B in eutopic and control were similar (P=0.46 and 0.42, respectively). Hence, the semi-quantitative RT-PCR analysis found that all three genes were upregulated in the ectopic endometrium.

We further investigated DNMTs expression using real-time quantitative RT-PCR, a more sensitive and accurate method than the semi-quantitative RT-PCR. We examined a total of 13 ectopic endometrial samples and 10 eutopic endometrial samples from endometriotic women with various menstrual phases. Eight normal endometrial tissue samples from endometriosis-free women were used as controls. Total RNA was isolated from laser captured epithelial cells. For the three groups, the proportion of samples taken at the secretory phase was similar, being 23.1%, 25.0%, and 20.0% in the ectopic endometrium, control, and eutopic endometrium groups, respectively. The mean age in these three groups was 36.0 (SD=6.42), 30.5 (SD=7.38), and 34.8 (SD=6.73) years old, respectively. Therefore, the three groups were fairly comparable in terms of age and menstrual phase.

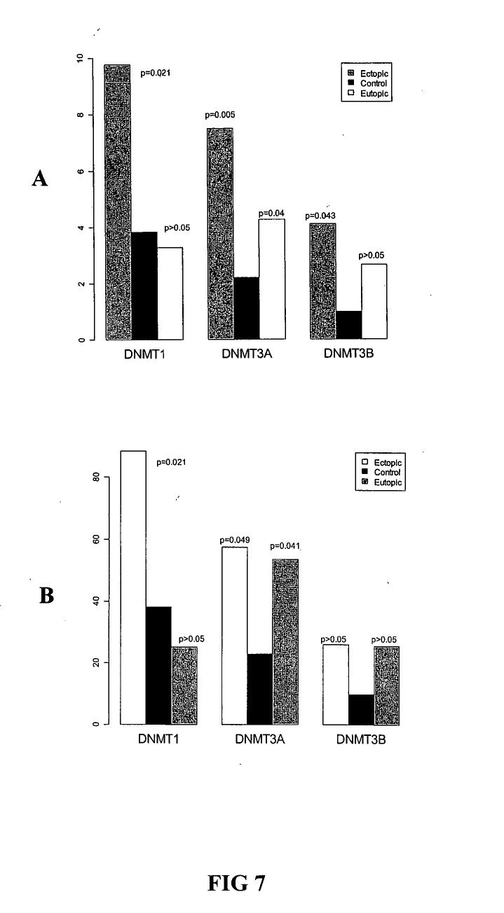

To adjust for possible difference in proliferation during different phases of the menstrual cycle, the expression levels of all DNMTs were normalized against proliferating cell nuclear antigen (PCNA) (Xiong et al. 2005 Gynecol Oncol 96:601-9). We found that relative expression of all DNMTs were significantly higher in ectopic endometrium as compared with controls, with p-values being 0.021, 0.005, and 0.043 for DNMT1, DNMT3A, and DNMT3B, respectively. The expression levels of DNMT1 and DNMT3B were similar in eutopic endometrium and controls (FIG. 7A). The DNMT3A expression level in eutopic endometrium of women with endometriosis was significantly higher than the controls. The fold change in ectopic endomerium as compared with controls was 2.54 for DNMT1, 3.40 for DNMT3A, and 4.16 for DNMT3B. For eutopic endometrium, the corresponding fold changes were 0.85, 1.93, and 2.70, respectively. Normalization using GAPDH as a reference standard yielded similar results (FIG. 7B), except for DNMT3B, in which there was no statistically significant difference between the ectopic endometrium and the control or between eutopic endometrium and the control. The fold change in ectopic endomerium as compared with controls was 2.33 for DNMT1, 2.50 for DNMT3A, and 2.67 for DNMT3B. For eutopic endometrium, the corresponding fold changes were 0.66, 2.33, and 2.60, respectively. The fold changes using PCNA normalization and GAPDH normalization were significantly correlated (r=0.83, p=0.041).

We also calculated and tested the statistical significance of Pearson's correlation coefficients of expression levels among the three genes. Using PCNA as a normalizing measurement, the correlation coefficients between DNMT1 and DNMT3A, between DNMT1 and DNMT3B, and between DNMT3A and DNMT3B were, respectively, 0.996 (p<2.2×10⁻¹⁶), 0.828 (p=3.0×10⁻⁸), and 0.817 (p=6.4×⁻⁸). Similar correlation coefficients, calculated based on GAPDH normalization, were 0.953 (p<2.2×10⁻¹⁶), 0.444 (p=0.012), and 0.431 (p=0.015), respectively. This indicated that the expression levels of these three genes are positively correlated.

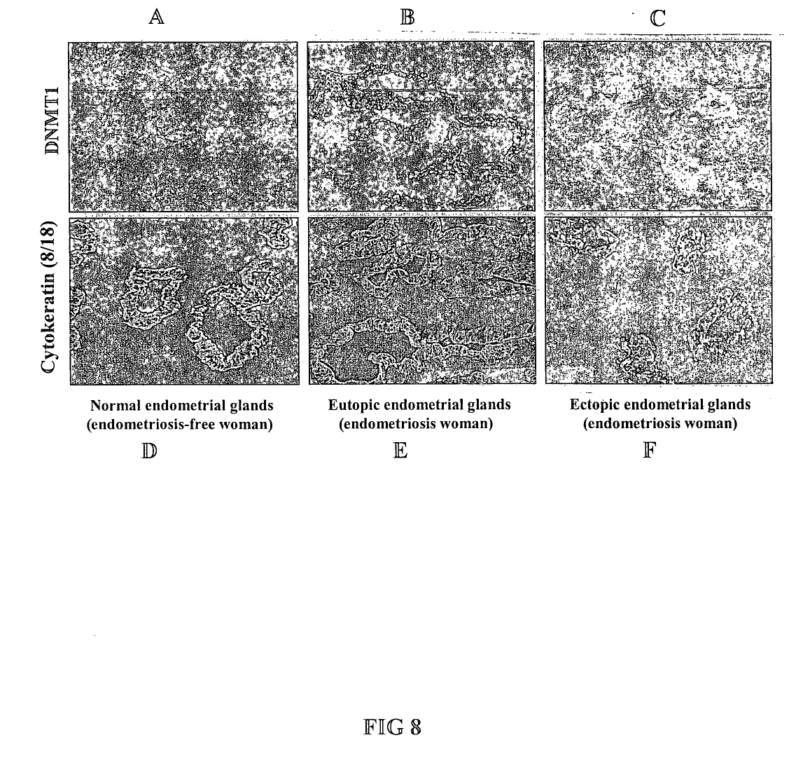

We also performed immunostaining to detect intracellular expression of DNMT1. FIG. 8 represented the results from control, eutopic and ectopic endometrium samples. While the control sample showed negative or focal weak staining for DNMT1, ectopic and eutpoic endometrial glands exhibited stronger or focally stronger staining for DNMT1. The result was consistent with quantitative RT-PCR analysis.

We have found that the expression levels of three genes coding for DNA methyltransferase DNMT1, DNMT3A, and DNMT3B are over-expressed in the ectopic endometrium as compared with normal controls or the eutopic endometrium of women with endometriosis. There was also indication that DNMT3A is upregulated in eutopic endomtrium of women with endometriosis. We also have found that DNMT1, DNMT3A, and DNMT3B expression levels are positively correlated with each other. Unfortunately, the limited amount of tissue samples available for laboratory analysis, after being halved for histological examination, severely limited our effort to carry out immunostaining analysis for more samples and for DNMT3A and 3B as well. It should be noted, however, that the gene expression of DNMT1, 3A, and 3B often correlates with their protein expression (Xiong et al. 2005 Cancer Res 65:2684-9). Our limited immunostaining study on DNMT1 is consistent with this notion.

Our results based on PCNA normalization and GAPDH normalization were overall consistent, but they differed for DNMT3B (p=0.043 in the former vs. p>0.05 in the latter). Factored in the semi-quantitative RT-PCR results, which were based on GAPDH, we believe that the difference is statistically different. This minor discrepancy between PCNA and GAPDH normalization has been observed in other studies (Xiong et al. 2005 Cancer Res 65:2684-9).

In endometriosis, gene dysregulation appears to be wide-spread and is responsible for most, if not all, phenotypic aberrations (Kao et al. 2003 Endocrinology 144:2870-81, Giudice et al. 2004 Endometriosis. Lancet 364:1789-99). Since endometriosis is a persistent disease, there must be cellular memory of some sort that constitutes a unique cell identity for endometriotic stromal or epithelial cells. Epigenetic regulation, especially through DNA methylation, is a flexible yet stable mechanism for maintaining such a cellular memory since methylation is heritable in somatic cells and largely irreversible.

As Rhee et al. demonstrated (Rhee et al. 2002 Nature 416:552-6), disruption of either DNMT1 or DNMT3B did not change gene-specific methylation and associated gene silencing in vitro. However, when both enzymes were disrupted, methyltransferase activity was nearly eliminated, causing extensive genomic demethylation. This study demonstrates convincingly that DNMT1 and DNMT3B together are responsible for most of the DNA methyltranserase activities in cancer cells, and presumably also in other cells. The positive correlation in gene expression levels among the three genes as demonstrated in this study indicates a common regulatory pathway as they may be synchronized to be upregulated in endometriosis. The simultaneous upregulation of all three genes in endometriosis appears to indicate an orchestrated action of both de novo and maintenance methyltransferases, which is likely to further result in aberrant methylation which, in turn, leads to persistent gene dysregulation in endometriosis.

In view of the aberrant expression of DNMT1, DNMT3A and DNMT3B, there is reason to believe that aberrant methylation may be more rampant than we previously thought. In conjunction with the evidence of aberrant methylation at HOXA10 in the eutopic endometrium of women with endometriosis (Wu et al. Am J Obstet Gynecol, 2005, 192) and at PR-B in the ectopic endometrium, the aberrant expression of the three DNMT genes strongly suggests that endometriosis is an epigenetic disease. As such, there is a hope that DNA demethylating agents may be employed to restore methylation aberrations (Miyamoto et al. 2005 Jpn J Clin Oncol 35:293-301). Along this line, identification of DNA methylation aberration as biomarkers for diagnostic and prognostic purposes is contemplated in accordance with the inventory, given the lack of any reliable biomarkers in endometriosis (Kitawaki et al. 2005 Hum Reprod 20:1999-2003).

Accordingly, applicants have demonstrated for the first time that DNMT1, DNMT3A, and DNMT3B are over-expressed in endometriotic tissues. This provides another piece of evidence that endometriosis may be ultimately an epigenetic disease. This may open up a new avenue for developing novel therapeutics for endometriosis and provide a potentially promising way for epigenetic reprogramming that restores dysregulated gene expression.

### Example 3

### Aberrant Methylation at HOXA10 May be Responsible for its Aberrant Expression in the Endometrium of Patients with Endometriosis

HOXA10 is a member of a gene family which contains a common, conserved region of 183 bp, called homeobox (Gehring WJ. Homeo boxes in the study of development. Science 1987; 236:1245-52). The gene family functions as transcription factors that regulate a plethora of other genes in development. In addition to its role in uterine development, hoxa10 expression has been shown to be important for uterine receptivity to implantation in mice. In humans, HOXA10 has been shown to be expressed in endometrium and to be regulated by estrogen and progesterone (Gui Y, et 1. Regulation of HOXA-10 and its expression in normal and abnormal endometrium. Mol Hum Reprod 1999; 5:866-73). Its peak expression occurs during the window of implantation, suggesting a possible role in uterine receptivity. In women with endometriosis which is known to be associated with subfertility (Olive D L, Schwartz L B. Endometriosis. N Engl J Med 1993; 328:1759-69), it has been shown that HOXA10 is aberrantly down-regulated in their endometrium during the secretory phase as compared to the control eutopic endometrium (Taylor H S, et al. HOX gene expression is altered in the endometrium of women with endometriosis. Hum Reprod 1999; 14:1328-31). The cause for this alteration still remains unknown. Accordingly, applicants investigate patterns of methylations in the transcriptional start sites of HOXA10.

Methods and Materials

Tissue Collection

Endometrial tissue samples were collected by pipelle suction curettage from 6 women, aged 33-36 years, with surgically and histologically confirmed stages III-IV endometriosis. For controls, endometrial biopsies were obtained from 4 women undergoing tubal ligations, aged 26-36 years, without endometriosis, confirmed at laparoscopy. In addition, endometrial tissues were isolated from the menstrual blood of 5 healthy women, aged 30-38 years, without any gynecologic complaints. The phase of menstrual cycle was determined according to the criteria of Noyes et al. Fertil Steril 1950; 1:3-25.). The endometrial tissue samples obtained from women with endometriosis and women who underwent tubal ligation were snap frozen on dry ice immediately after surgical dissection and then stored in a −80° C. freezer. Menstrual blood was stored in freezers within 30 minutes after collection on sanitary napkins and later transferred to a −80° C. freezer. The research was approved by the Institutional Review Board of MCW.

Methylation Specific PCR (MSP)

All samples were initially screened by MSP at HOXA10 which is about 3.71 kb and its structure is schematically depicted in FIG. 9. The sequence of the HOXA10 gene is well known in the literature (as defined by Accession NM_{—}018951, MIM: 142957 and GeneID: 3206). Using the MethPrimer software (Li L C, Dahiya R. MethPrimer: designing primers for methylation PCRs. Bioinformatics 2002; 18:1427-31) (downloadable), we identified three CpG-rich fragments in HOXA10, one (F1) in the 5′ upstream of exon 1, and two (F2 and F3) in the intronic region sandwiched by exons 1 and 2.

Sixty ng of bisulfite-modified DNA was used for each MSP assay. The primers designed for F1 and F2 fragments were as follows: 1) F1 fragment, methylated: forward 5′-GTTT TTATAGTTTTCGGTTTTCGG-3′ (SEQ ID NO: 26), and reverse 5′-ACTCCCAATTTAATTTCGTAAA CG-3′(SEQ ID NO: 27); unmethylated: forward 5′-TTTTTATAGTTTTTGGTTTTTGG-3′ (SEQ ID NO: 28), and reverse 5′-CACTCCCAATTTAATTTCATAAACAC-3′ (SEQ ID NO: 29). 2) F2 fragment, methylated: forward: 5′-ATAAGTTTATTAATCGGCGAAGTTC-3′ (SEQ ID NO: 30), and reverse 5′-AATAAAAA AAACA AAAAAAACCGAT-3′ (SEQ ID NO: 31); unmethylated, forward 5′-AAGTTTATTAATTGGTGAAGTT TGA-3′ (SEQ ID NO: 32), and reverse 5′-CCAATAAAAAAAACAAAAAAAACCA-3′ (SEQ ID NO: 33). The primers were designed using the MethPrimer program.

Genomic DNA extracted from peripheral blood of healthy individuals was aliquoted into two parts: one was treated with methylase SssI (New England Biolab, Beverly, Mass.) as a positive control, and the other received no treatment and was served as a negative control. After methylase treatment, the positive control would have 100% methylated cytosines in all cytosine sites.

Bisulfite Sequencing of HOXA10 Gene

The methylation statuses were further validated by bisulfite sequencing, which is considered to be the gold standard for methylation evaluation (Frommer M, et al. PNAS USA 1992; 89:1827-31.). Genomic DNA extracted from endometrial tissues was modified with sodium bisulfite following the manufacturer's instructions (Zymo Research, Orange City, Calif.). This modification resulted in a conversion of unmethylated cytosine to thymine, whereas the methylated cytosine remained unchanged. A total of 60 ng of bisulfite-modified DNA was subjected to PCR amplification and directly sequenced using the ABI 3700 automated sequencing system (Applied Biosystems). The primers for bisulfite sequencing were designed to complementary to DNA fragments containing no CpG dinucleotides, thus allowing unbiased amplification of methylated and unmethylated alleles. The following primers were designed to amplify CpG rich regions within the HOXA10 gene: 1) F1 fragment: forward, 5′-GTTGGGGTAGTTTTTATAGTTTT-3′ (SEQ ID NO: 34), and reverse, 5′-ATAA CCCTTTCTAACTAACATTTCTTATAC-3′ (SEQ ID NO: 35); 2) F2 fragment: forward 5′-GGA GGAT TTAGAGT TTTGGTTTTT-3′ (SEQ ID NO: 36), and reverse 5′-AACCCTTCTCTAACTCTCAA CTCAA-3′ (SEQ ID NO: 37); 3) F3 fragment: forward 5′-TGTGATTTGGGATATTT TTTTTATG-3′(SEQ ID NO: 38), and reverse 5′-AAAACTCC TTCTCCAACTCCAATAT-3′ (SEQ ID NO: 39).

Quantitative Real-Time RT-PCR

O.C.T (Sakura Finetek, Torrance, Calif.) embedded-frozen endometrium tissue sections were stained using Histogene LCM Frozen Section Staining solutions (Arcturus, Mountain View, Calif.). The glandular epithelial cells were dissected by laser capture microdissection. Total RNA was extracted from the captured epithelial cells using the Picopure RNA isolation kit (Arcturs) and treated with RNase-free DNase I (Invitrogen). The total RNA (10 ng) was then reverse-transcribed (Superscript II Reverse Transcriptase, Invitrogen) and the HOXA10 mRNA amounts were quantified by a real-time RT-PCR analysis using SYBR Green I detection kit (Qiagen). PCR reactions were carried out on the ABI PRISM 7900HT Sequence Detection System. The amount of HOXA O mRNA of each subject was normalized with that of 18S mRNA (endogenous control, Ambion) taken from the same subject and the relative amount of HOXA10 mRNA was calculated using relative quantitation of gene expression (Applied Biosystems). The primers selected for RT-PCR yielded 210 bp PCR product from HOXA10 mRNA. The HOXA10 mRNA primers were: 5′-GCCCTTCCGAGAGCAGC AAAG-3′ (SEQ ID NO: 40) and 5-AGGTGGACGCTGCGGCTAATCTCTA-3′ (SEQ ID NO: 41). The RT-PCR products were checked on a 0.8% agarose gel.

Data Analysis

The methylation patterns of each subject were listed along CpG sites sequenced for a given region. For each CpG site, no methylation was coded as 0, partial methylation, 0.5, and complete methylation, 1. Partial methylation occurred when equal or nearly equal amounts of C and G were present in the sample, manifested as an identical or nearly identical peak in the SEQUENCER, thus precluding an unambiguous base calling. For each subject, scores across all sequenced CpG sites in the same region were added. The difference in scores between the cases and the controls was compared and evaluated by a randomization test by adding scores in a group (case or control). 100,000 permutations were conducted to arrive the empirical p-value,

$$p = \frac{{\sum }_{i} {I}_{i}}{n} ,$$

where n is the number of permutations, and Iᵢ is the indicator variable indicating whether or not the total score Sᵢ in the patient group at the ith permutation exceeds the observed score S₀. For gene expression data, a one-sided t-test was used. All computations were carried out with R (version 1.9.0).

Results

In MSP assay of 14 endometrial tissue samples (6 cases, 3 tubal ligation controls and 5 menstruum controls) at F2, both methylated and unmethylated bands were observed in all 6 endometriosis patients but only one control. All other 7 controls showed only unmethylated bands (FIG. 10). The MSP assays of F1 fragment yielded similar result (data not shown). For F3, the MSP assay was not performed.

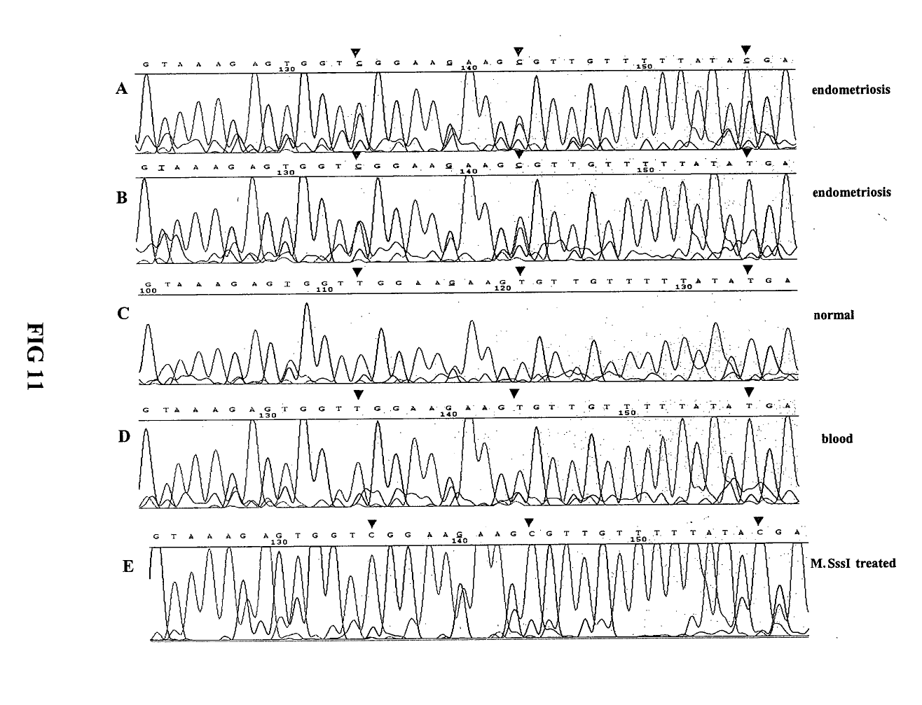

The MSP results were further validated by bisulfite sequencing. The representative result is shown in FIG. 11, where samples from endometriosis patients showed both C and T in all three CpG sites. In contrast, only T's were present in normal controls. The result also indicated partial methylations occurred in patients with endometriosis (FIG. 11).

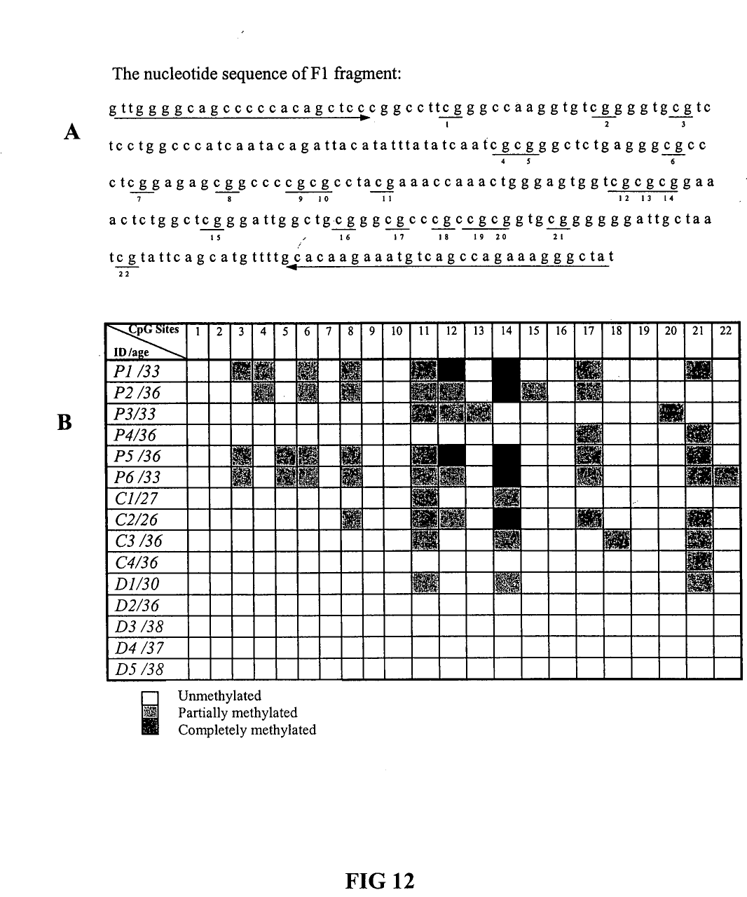

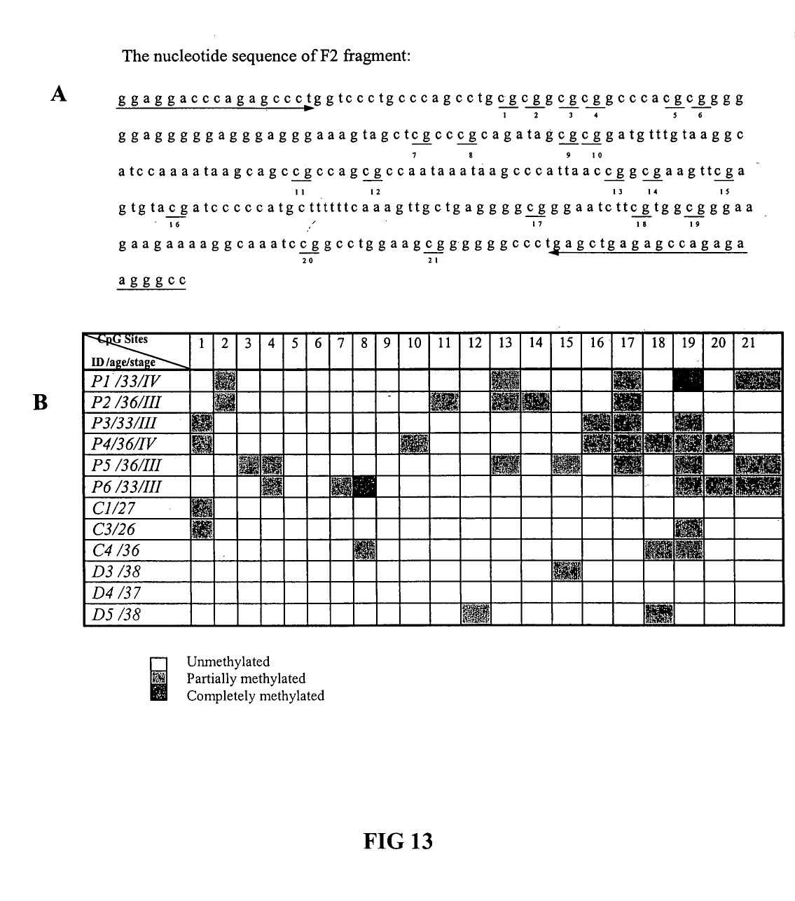

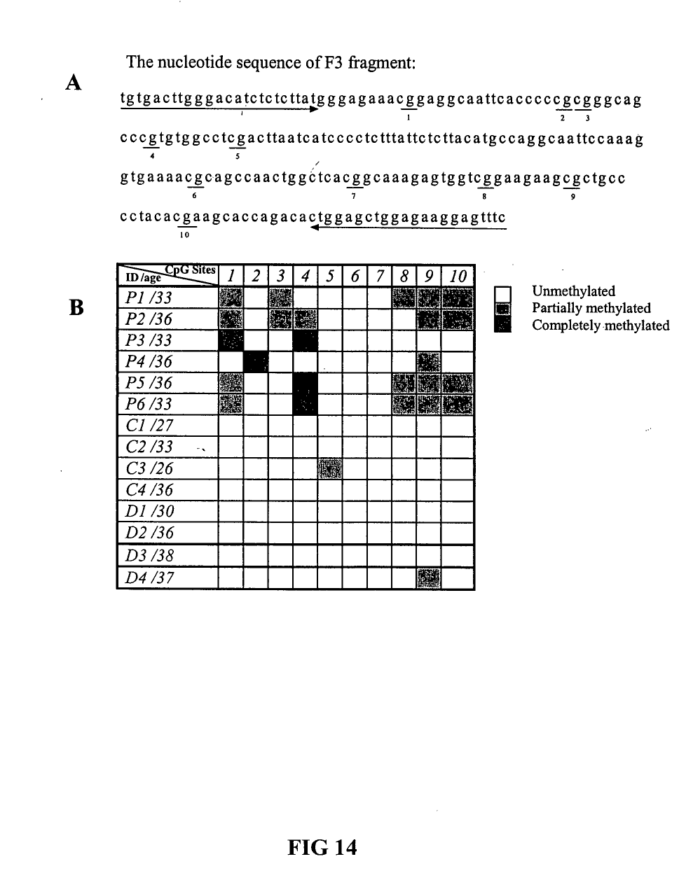

The results of the bisulfite sequencing of F1, F2 and F3 fragments were presented schematically in FIGS. 12, 13 and 14. As shown in the FIG. 12, F1 fragment contains 22 CpG sites and all samples were successfully sequenced. The randomization test based on 100,000 permutations resulted in a P value less than 0.0001, which is highly significant. FIG. 13 demonstrates the methylation patterns of the F2 fragment of 12 individuals after bisulfite sequencing. Three individuals (C2, D1 and D2) were not shown in the figure due to failed PCR amplifications. The pattern was statistically highly significant, and extremely unlikely due to chance (P<0.00001, based on 100,000 permutations). For the F3 fragment, 14 samples were successfully sequenced, and the results were shown in FIG. 14. The randomization test based on 100,000 permutations resulted in a P value of less than 0.0001. These results indicate that HOXA10 was hypermethylated in the three fragments examined in the endometrium of women with endometriosis as compared with that of women without.

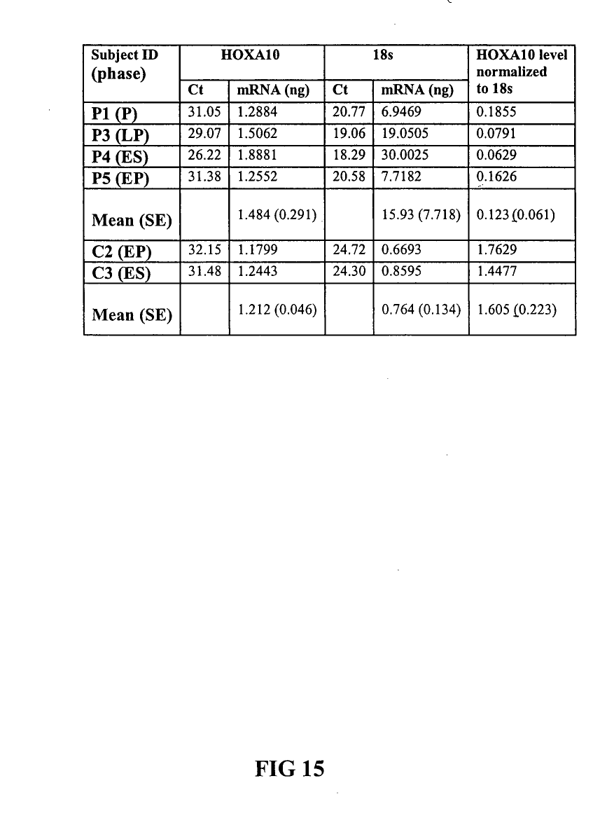

For 4 women with endometriosis and 2 without, from whom total RNAs were available, the expression of HOXA10 was evaluated by quantitative real-time RT-PCR (FIG. 15). In two women without endometriosis, the expression level of HOXA10 gene was significantly higher than that of 4 women with endometriosis (P=0.0295, one-sided t-test test). This agreed with the finding by Taylor et al. (Hum Reprod 1999; 14:1328-31).

Discussion

As a homeodomain transcription factor, HOXA10 is dynamically expressed in uterine endometrium where it is necessary for embryo implantation. In human endometrium, HOXA10 is expressed in both epithelial and stromal cells, and the expression has been shown to be driven by sex steroids, with peak expression occurring in the mid-secretory phase, paralleling with rising progesterone levels during the same stage. In women with endometriosis, the endometrium failed to show the expected mid-secretory rise in HOXA10 gene expression and its cause remains unclear. Most women with endometriosis show normal sex steroids production and it has been showed that the estrogen receptor (ER) and progesterone receptor (PR) activities in the eutopic endometrium of women with endometriosis were no different from those of women without the disease (Jones R K, Bulmer J N, Searle R F. Hum Reprod 1995; 10:3272-9), although in the latter the results appeared to be mixed (Leyendecker G, et al. Hum Reprod 2002; 17:2725-36). Hence a question naturally arises as what causes the apparent down-regulation of HOXA10 in the endometrium of women with endometriosis. It should be noted that while both ER and PR activities in epithelium diminish sharply in mid and late secretory phases, their activities in stroma are somewhat preserved. In addition, the expression of aromatase P450, which synthesizes estrogen, has been reported to be increased in the eutopic endometrium of women with endometriosis. Since sex steroids induce HOXA10 expression in the endometrium, its aberrant expression may be attributable to either aberrant local production of steroids and altered, perhaps cell-specific, ER and/or PR activity level in the endometrium. These observations suggest a possibly complex mechanism in regulating HOXA10 gene expression in both epithelium and stroma, for example, PR or ER induction of HOXA10 expression in epithelium as a paracrine event mediated by the stroma. Even if this is indeed the case, one question still lingers: What, then, causes the changes in either local production of sex steroids or the activities of their receptors? Since most endometriosis, once diagnosed, does not go away without any intervention, there must be some mechanism that maintains these aberrations that result in aberrant expression of HOXA10.

Applicants report here an entirely different and biologically plausible mechanism for the observed down-regulation of HOXA10, that is, aberrant methylation may be responsible for aberrant gene expression. As it has become abundantly clear, methylation of promoter regions is associated with a compacted chromatin structure, accompanying transcriptional silencing of the affiliated gene. Methylation-induced epigenetic changes play an important role in development, and can also occur with age. Since methylation changes are heritable in cell divisions, epigenetic modification of the genome through methylation provides a mechanism for maintaining stable gene activity states. In addition, since methylation states are reversible, they can be modified by environmental or lifestyle factors, which, in turn, may contribute to the development of abnormal phenotype.

Given that hypermethylation in the promoter regions tend to suppress the gene expression, and that methylation provides a mechanism for maintaining stable gene activity states, there is reason to believe that the aberrant expression at HOXA10 in the endometrium of women with endometriosis may result from aberrant methylation. In addition, that both HOXA10 and methylation are involved in development, that methylation changes with age and that age is the only sociodemographic risk factor consistently reported for endometriosis, all these observations appear to suggest that it is not only biologically plausible but also highly likely that aberrant methylation at HOXA10 may be responsible for its aberrant expression. Lastly, our study has been confined to patients with stages III-IV endometriosis. It is believed that applicants results hold up for early stages of endometriosis.

It is noted that applicants did not evaluate methylation patterns in matched endometriotic tissues because the interest in HOXA10 has been mainly in its role in endometrial development and in implantation. So far there has been no report on its expression levels in ectopic endometrium. Since the main focus was to provide a biological plausible explanation to possible mechanisms underlying aberrant expression of HOXA10 in the endometrium of women with endometriosis, applicants only evaluated eutopic endometrium. HOXA10 gene may or may not have aberrant expressions or even aberrant methylation, as with many other genes. Even if there are aberrant expressions or aberrant methylations, it is possible that these changes may be a consequence of endometriosis.

Epigenetic effects by means of DNA methylation have been demonstrated to have important roles in development, tumorigenesis, and other diseases. To our knowledge, this is the first report that the HOXA10 gene in the endometrium from women with endometriosis has aberrant hypermethylations, and suggests that endometriosis, like neoplasia, may also be an epigenetic disease.

Notably, for detection of methylation changes of genes associated with endometriosis, menstrual blood can be a valuable, abundant, and non-invasive source (see, Fiegl et al., Methylated DNA collected by tampons—a new tool to detect endometrial cancer. Cancer Epidemiol Biomarkers Prev 2004; 13:882-8). Since aberrant methylation at HOXA10 or other genes in endometrium are believed to be a cause for their aberrant expressions, which, in turn, are responsible for abnormal uterine receptivity and implantation capability, menstrual blood can be used for screening and diagnosis.

The partial methylation at HOXA10 observed by applicants could be due to cell type heterogeneity (stroma, epithelium, etc.) or genuine methylation heterogeneity or both. One way to resolve this is by selective examination of different cell types through, say, laser capture microdissection (LCM). However, harvesting sufficient cells by LCM enough for bisulfite sequencing without introducing further heterogeneity could pose a big challenge that needs to be resolved in future studies. In addition, a more definite proof that aberrant methylation at HOXA10 is the cause for its aberrant expression in the endometrium of women with endometriosis also awaits future studies.

### Example 4

### Inhibition of Proliferation of Endometrial Stromal Cells

As discussed herein current major medications in treating endometriosis are effective in treating pain, most likely through suppression of proliferation of the implants, yet their effectiveness is relatively short-term and they all have many undesirable, and sometimes severe, side effects. In an effort to identify more effective medications in treating endometriosis, using a recently established immortalized endometrial stromal cell line, we carried out cell proliferation assays for cells treated with trichostatin A (TSA), RU486, CDB-2914, and N-acetylcysteine, and ICI 182780. Gene expression levels for PR-A, PR-B, AR, Fas and FasL were measured. Protein expression levels for ERα, ERβ, and AR were also measured.

The results of the cell proliferation assay for NAC, H2O2, CDB, and RU486 indicates that all of these compounds had various antiproliferative effects. TSA had more potent and longer lasting antiproliferative effect than CDB and NAC, even in the presence of an oxidant, H2O2. Its antiproliferative effect was concentration-dependent. PR-A, PR-B, AR, and FasL expression were all increased as compared with untreated cells. Thus, the cell line appears to be an adequate model for stromal components of endometriotic implants. The upregulation of PR-B and AR may be responsible for antiproliferative effects induced by TSA, a histone deacetylase inhibitor (HDACI).These result support the notion that HDACIs may be a promising therapeutics in treating endometriosis due to their antiproliferative effects as well as the potential to restore gene dysregulation through chromatin remodeling.

Chemicals and Reagents

Chemicals and reagents utilized in the experiments below include: TSA (Sigma), RU 486 (Sigma), ICI 182780 (Tocris, Avonmouth, UK), CDB (HRA Pharma, Paris, France) each dissolved in absolute ethanol to make 10 mM stock solutions. NAC (Sigma) was dissolved in PBS to make 600 mM stock solution. All stock solutions were aliquoted and stored in −20° C. H202 stock solution was purchased from Sigma. All compounds were further diluted in the culture media to achieve desired concentrations.

Cell Line and its Maintenance

An immortalized human endometrial stromal cell line was maintained following Krikun et al. (Krikun et al. 2004 Endocrinology 145:2291-6). Cells were maintained in phenol red-free DMEM/F12 (Gibco, CA) medium supplemented with 10% charcoal-dextran stripped FBS (Biomeda, CA), 1 μl/ml sodium pyruvate (Gibco, CA) and 1× Pen/Strep/Fungizone (Gibco, CA), at 37° C. with 5% C02 in a humidified incubator. The cell line, referred to as the Yale Human Endometrial Stromal or YHES cell line hereafter, has been shown to be karyotypically, morphologically, and phenotypically similar to the primary parent cells.

Drug Treatment and Cell Proliferation Assay

The YHES cells were harvested by trypsinization and seeded in 96-well plate at ˜1×104 cells/well. The cells were allowed to attach to the plate for overnight at 37° C. with 5% C02 in a humidified incubator. Then they were pretreated with physiological concentration of E2 (10-9M) and P (10-7M) for 24 hr to maintain hormonal responsiveness. Following the pretreatment, cells were treated with 1) ethanol alone as solvent control (untreated control); 2) TSA at concentration of 100 μM, 10 μM, 1 μM, 0.1 μM, respectively; 3) RU486 at concentration of 100 μM, 10 μM, 1 μM, 0.1 μM, respectively; 4) ICI at concentration of 100 μM, 10 μM, 1 μM, 0.1 μM, respectively; 5) CDB at concentration of 100 μM, 10 μM, 1 μM, 0.1 μM, respectively; 6) NAC at concentration of 10 mM and 30 mM, respectively; 7) H202 at concentration of 100 μM, 10 μM, 1 μM, respectively, for 48 hr. Cell growth was valuated by the MTT assay (ATCC, Manassas, Va.). MTT assay was performing according to the manufacturer's instructions, and in triplicate to generate mean values. Cell numbers in the untreated control were arbitrarily assigned as 1. The relative cell growth was expressed as fold increase/decrease relative to the control. Data were presented as mean values from three independently repeated experiments.

RNA Isolation and cDNA Synthesis

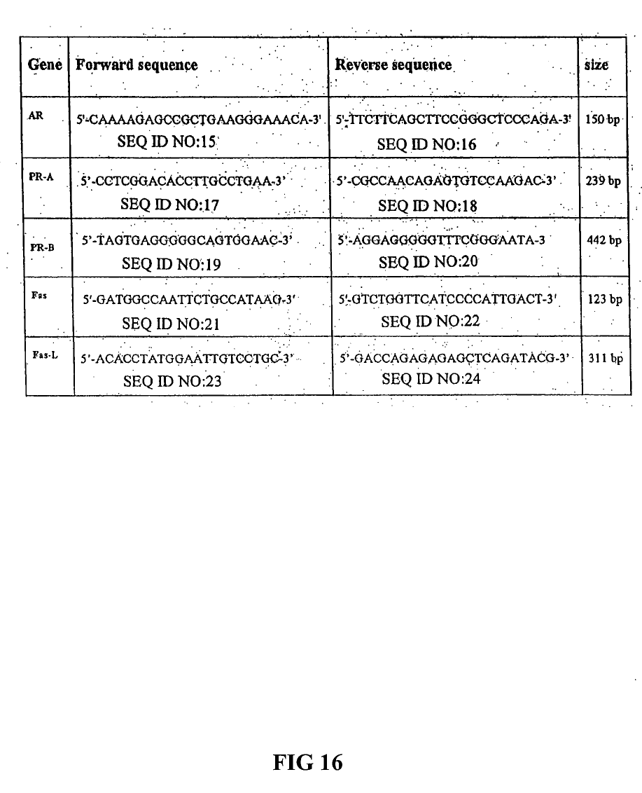

The YHES cells were harvested by trypsinization and seeded in 12-well at 5×104 cells/well plates and were pretreated with E2 and P as described above. Cells were then treated with TSA (10 μM), CDB (100 μM), NAC (10 mM) and ethanol alone for 8, 18 and 36 hours. Total RNA was isolated from treated cells using TRIzol reagent (Invitrogen, Carlsbad, Calif.) according to the manufacturer's instructions. The integrity of total RNA was inspected on an agarose gel. One microgram of total RNA from each sample was treated with DNase I to remove potential DNA contamination and then reverse-transcribed using Superscript II Reverse Transcriptase (Invitrogen). First-strand cDNA obtained from RNA samples was stored at −80° C. until use. To measure the mRNA expression levels of AR, PR-A, PR-B, Fas and FasL in each cell culture, semi-quantitative PCR analysis was performed. The cDNA primers designed for subsequent PCR were listed in FIG. 16.

Real-Time RT-PCR

Quantitative real-time RT-PCR was carried out on an ABI 7900 DNA sequencer (Applied Biosystems), and monitored by SYBR Green I (Qiagen, Valencia, Calif.). The PCR products of the expected size were visualized on a 0.8% agarose gel. The relative mRNA level of each gene was calculated using relative quantitation of gene expression (Applied Biosystems, Foster City, Calif.) with 18s as an endogenous control. The reproducibility of the quantitative measurements was evaluated by two independent cDNA syntheses. The means and standard deviations of the replicated measurements were calculated and presented as mean ±s.d.

Western Blot Analysis

Western blot analysis was performed to measure protein expression of β-actin, ERα, ERβ, and AR. Cells were harvested by trypsinization and seeded in 6-well plates at 2×104 cells/well and were pretreated with E2 and P as described above and followed by treatment with TSA (10 μM), CDB (100 μM), ICI (100 μM), NAC (10 mM), or ethanol alone for 24 hr. Whole cell lysate was prepared as previously described (44) by using RIPA lysis buffer containing 50 mM Tris, pH 7.4, 150 mM NaCl, 12 mM EDTA, 1% Triton X-100, 0.5% sodium deoxycholate, and 0.1% SDS. To avoid degradation, a protease inhibitor cocktail III (Sigma) was added at 2%. Total protein was quantified using a BCA protein assay kit (Pierce, Rockford, Ill.). Ten microgram of total protein was separated by electrophoresis on 10% SDS-polyacrylamide gels. The protein was then transferred onto PVDF membranes (Milipore, Bedford, Mass.). To inhibit nonspecific binding, the membrane was initially blocked with 5% nonfat dry milk in PBS for 1 hr at room temperature. The blot was then subsequently incubated with primary antibody rabbit anti-human ERα (Santa Cruz Biotechnology, CA), rabbit anti-human ERβ (Zymed Laboratories, South San Francisco, Calif.), rabbit anti-β-actin (Cell Signaling Technology, Beverly, Mass.), or rabbit ant-human AR (Santa Cruz Biotechnology, CA), respectively, in blocking solution (5% nonfat dry milk plus PBS) for 2 hr at room temperature. After washing with PBS plus 0.5% Tween-20, the membranes were then incubated with the secondary antibody goat anti-rabbit IgG (H+L)-HRP (Santa Cruz Biotechnology, CA) at 1:8000 dilution in blocking solution for 2 h at room temperature. After washing, membrane blot was developed using the enhanced chemiluninescence (ECL) protocol (Amersham Pharmacia Biotech).

Statistical Analysis

Data were expressed as the mean of two to four experiments, each in triplicate samples for individual treatments or dosage regimens. Two-sided Student's t-test was used to test whether the means of the number of viable cells in the two tested groups are equal. Values were presented as the means.d. Statistical tests were considered to be statistically significant if P<0.05.

Results

Expression of ERα and ERβ in the HES Cell Line

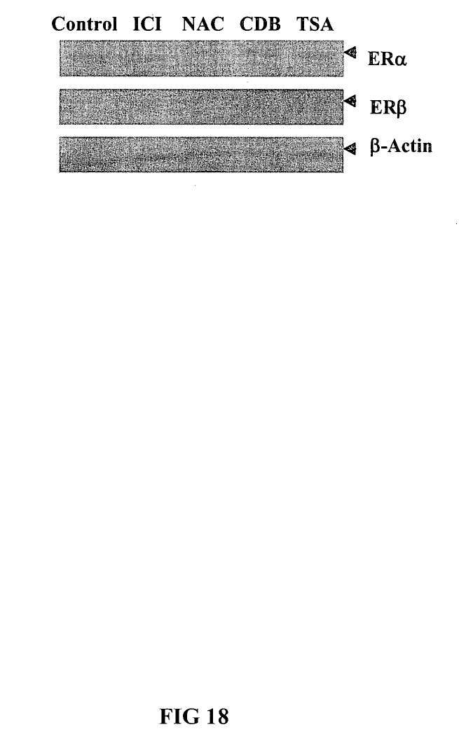

The immortalized YHES cell line has been shown to have normal progestational response and to be karyotypically, morphologically, and phenotypically similar to the primary parent cells (Krikun et al. 2004 Endocrinology 145:2291-6). As part of the validation, we determined its estrogen receptor α (ERα) and β (ERβ) expression. We pretreatedcells with a physical concentration of E2 (10-9M) and P (10-7M) for 24 hours. As pointed out by Catalano et al. 2003 Mol Hum Reprod 9:465-73, these steroid concentrations have been shown to elicit responses in endometrial explant cultures and closely resemble plasma concentrations reported during the mid-secretory phase of the endometrium. Whole cell lysates were prepared after 24 hours of treatment with either TSA, CDB, NAC, ICI, or vehicle only (ethanol, as control). As seen in FIG. 18, ERα was expressed on all treated cells and untreated control cells while ERβ was only weakly expressed in all groups. This is consistent with the well-documented observation that ERβ is expressed predominantly in glandular epithelial cells and that ERα expression is higher than ERβ in endometrial stromal cells (Matsuzaki et al. 1999 Mol Hum Reprod 5:559-64, Mylonas et al. 2005 Anticancer Res 25:1679-86), in the ratio of ˜16:1. Based on studies on ERα- and ERβ-knockout mice (Krege et al. 1998 Proc Natl Acad Sci USA 95:15677-82), we concluded that this cell line is estrogen responsive.

Effects of H2O2, NAC, RU486 and CDB on Cell Proliferation

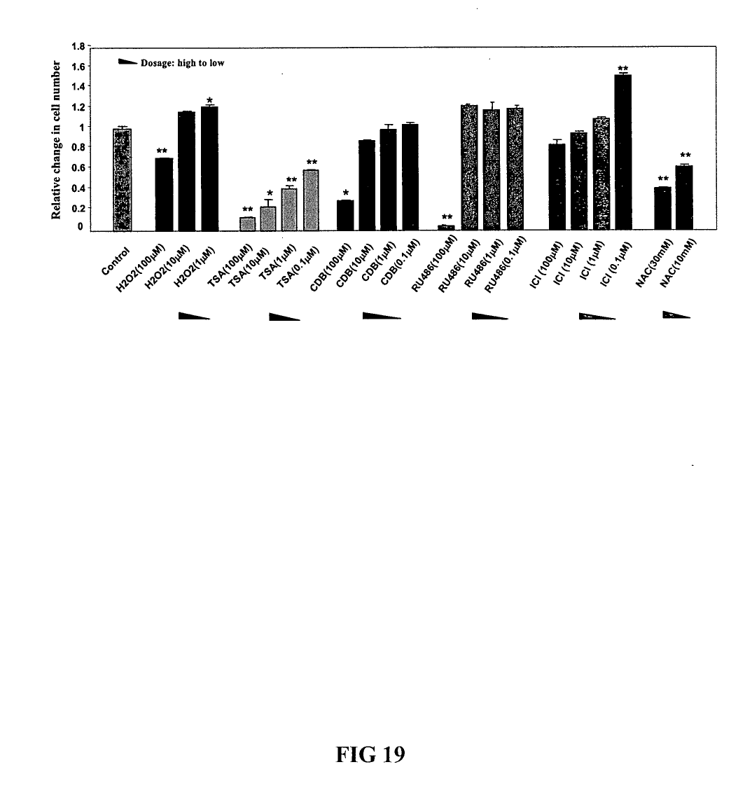

We sought to assess the effects of H2O2, NAC, RU486, and CDB on the YHES cell proliferation, since their effects on endometrial cell growth have been well documented either in vivo or in vitro. Treatment with H202 at low concentration of 1 μM and 10 μM increased cell proliferation by 18% and 22%, respectively, while treatment with higher concentration of 100 μM of H202 induced inhibition of proliferation (FIG. 19). In addition, treatment with antioxidant NAC at concentrations of 10 mM and 30 mM resulted in significant suppression of proliferation by 35% to 50%, respectively, a result identical to that of reported (Foyouzi et al., 2004 Fertil Steril 82 Suppl 3:1019-22). For CDB, the highest concentration resulted in significant suppression of cell proliferation. At other concentrations, the treatment elicited seemingly dose-response yet statistically non-significant inhibition of cell proliferation. This seemed to be consistent with observation that SPRMs, such as J867, have antiproliferative effects (Chwalisz et al. 2000 Steroids 65:741-51).

The effect of RU486 on cell proliferation appeared to be entirely concentration-dependent (FIG. 19). Ten-fold difference in concentration (100 μM vs. 10 μM) resulted in completely different effects: at highest concentration of 100 μM, RU486 reduced cell proliferation by >95% as compared with controls. In contrast, at concentration of 10 μM or lower, there was no inhibition of proliferation at all. This appeared to be consistent with the observation that the effect of RU486 on endometrial cell proliferation depends largely on dosage and other factors (Chabbert-Buffet et al. 2005 Hum Reprod Update 11:293-307). Based on these findings, it appeared that the YHES cell line is an appropriate model for the stromal components of endometriotic implants.

Effects of TSA and ICI on Cell Proliferation

With the assurance of the appropriateness of the cell line in the evaluation of induced proliferation/antiproliferation in endometrial (and hopefully endometriotic) stromal cell, we then assessed the effect of TSA and ICI on cell proliferation. For cells treated with TSA, there was a strong concentration-dependent antiproliferative effect, with the strongest proliferation suppression by ˜90%, as compared with the control, occurred at the highest concentration of 100 μM. The effect of antiproliferation in all four concentrations was statistically significant. For cells treated with ICI, there was no statistically significant reduction in proliferation, although there appeared to have a concentration-dependent effect (FIG. 19). In fact, at the lowest concentration of 0.1 μM, there was about 40% increase in proliferation as compared with the control group.

Effects of TSA, CDB, RU486, ICI and NAC on H2O2-Induced Cell Proliferation

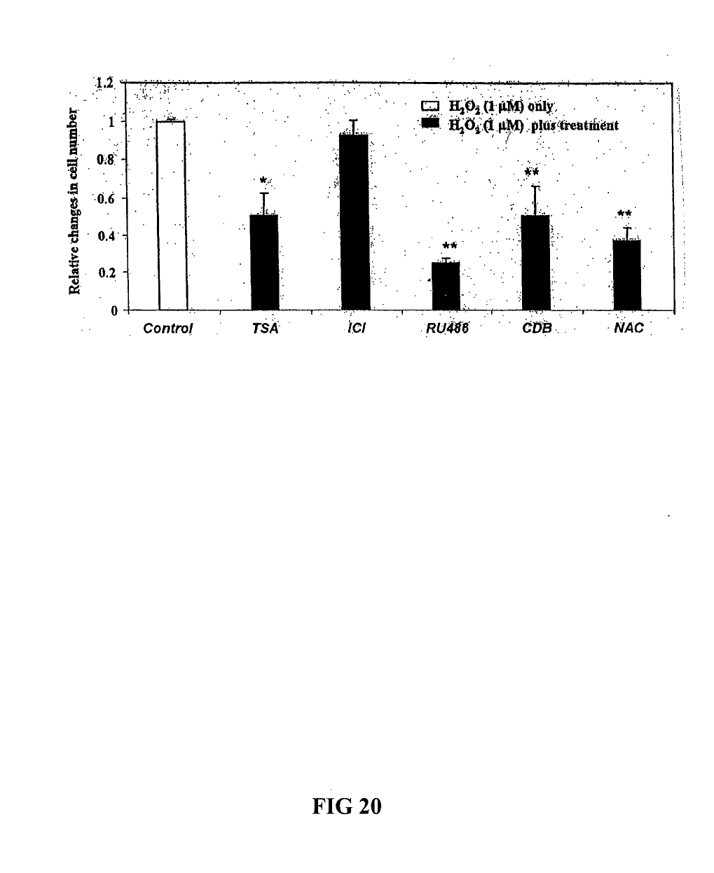

In this experiment, we assessed the antiproliferative effects of NAC, CDB, RU486, and TSA in the presence of an oxidant agent, H2O2, at the concentration of 1 μM, along with physiological concentration of E2 and P as described previously. The addition of H202 was meant to mimic the oxidative stress as reported in endometriosis. As expected, NAC, at 10 mM concentration, significantly suppressed the H2O2-induced proliferation by 63% as compared with the cells treated with H202 only (FIG. 20). RU486 suppressed cell proliferation by 75% at the concentration of 100 μM. CDB, at 100 μM, significantly suppressed the cell proliferation by 49%. Similar to CDB, TSA significantly suppressed H2O2-induced cell proliferation by 50% at 100 μM. Interestingly, ICI did not significantly inhibit H2O2-induced cell proliferation. The experiment indicated that NAC, CDB, RU486 and TSA, but not ICI, suppressed cell proliferation in the presence of the oxidant H202.

Long-term Proliferation Suppression by TSA, NAC, RU486, and NAC

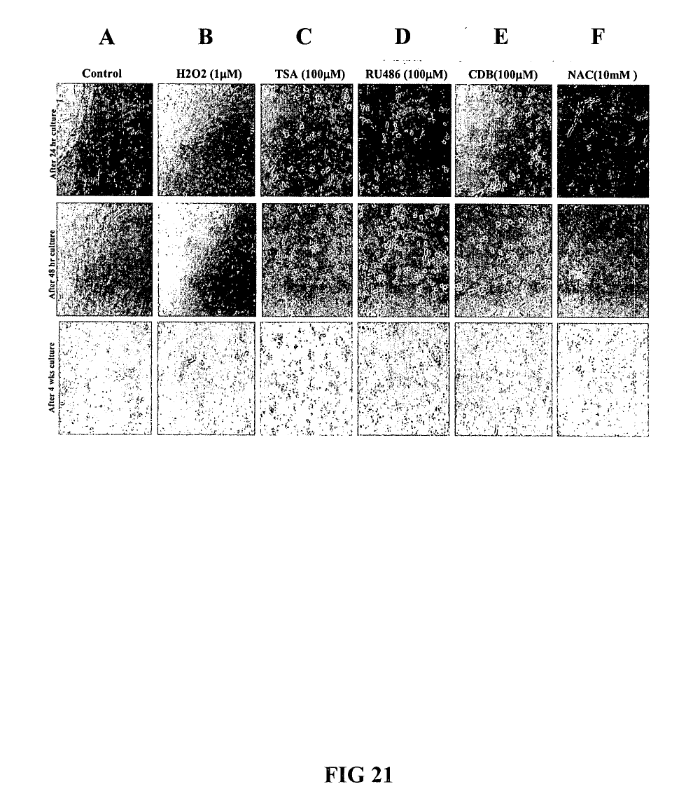

After treatment with TSA and CDB, we continued culturing the cells for up to 4 weeks, with the culture medium changed every 10 days. Intriguingly, we found that cells treated with CDB and NAC apparently recovered from apoptosis only 8-10 days after treatment while cells treated with TSA (at 100 μM) or RU486 (at 100 μM) showed no sign of recovery (FIG. 21). In fact, 24 h after the treatment, cells in the TSA, RU486 and CDB groups all showed signs of apparent apoptosis, characterized by the condensation of nucleus (FIG. 21). The apparent apoptosis appeared to be more pronounced 48 h after the treatment (FIG. 21). Four weeks after the treatment, however, cells treated with CDB and NAC apparently recovered as judged by their morphology since very few cells showed any sign of apoptosis and the cell population appeared to be increased (FIG. 21). In contrast, the TSA and RU486 groups did not recover, retaining the dormant morphology with few viable cells (FIG. 14). This suggested that the antiproliferative effect of TSA or RU486 (at high concentration) may be longer lasting than CDB and NAC.

Effects of TSA, CDB, and NAC on Gene Expression of AR, PR, Fas and FasL

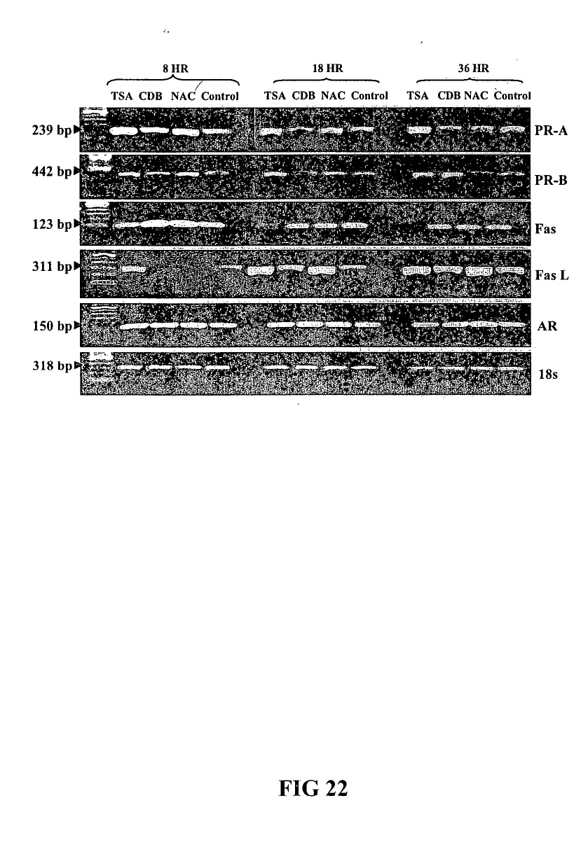

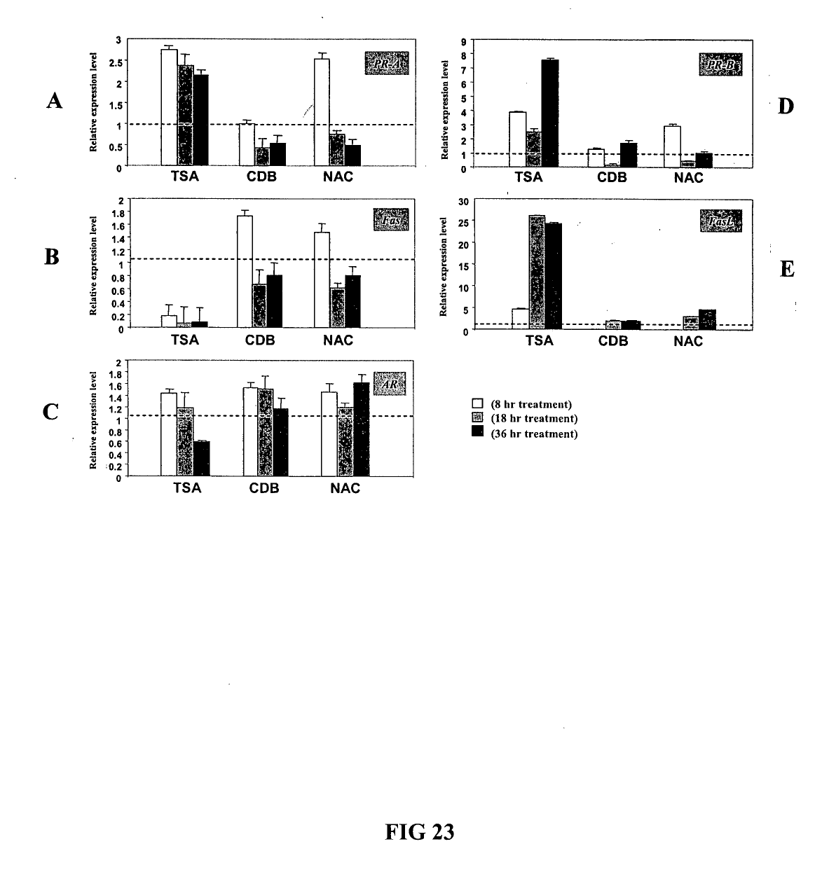

We measured mRNA levels of AR, PR-A, PR-B, Fas and FasL in the cells treated with TSA, CDB, NAC as well as in untreated control cells. We found that the expression levels of AR, PR-A, and PR-B were all increased for all groups after 8 h, 18 h, and 36 h of treatment (FIG. 22). The results were further confirmed by real-time RT-PCR analysis (FIG. 23). Overall, the TSA group had the greatest increase in PR-A and PR-B expression, while its increase in AR expression was similar to CDB and NAC groups after 8 h and 18 h of treatment. After 36 h of treatment, the AR expression in the TSA group appeared to be decreased by about 50% as compared with the control group, while the CDB and NAC groups maintained the increased expression (FIG. 23). For Fas/FasL, the CDB and NAC groups had the increased Fas expression 8 h after the treatment, but the expression decreased 18 and 36 h after the treatment. Their FasL expression levels increased by about 2-4 fold 18 and 36 h after the treatment. In contrast, cells treated with TSA showed dramatic increase in FasL expression, especially 18 and 36 h after the treatment (FIG. 23). However, its Fas expression appeared to lower than the control group. These observations suggest that TSA may have a different antiproliferation mechanism than that of CDB or NAC.

Effects on Protein Levels of AR, ERα, and ERβ

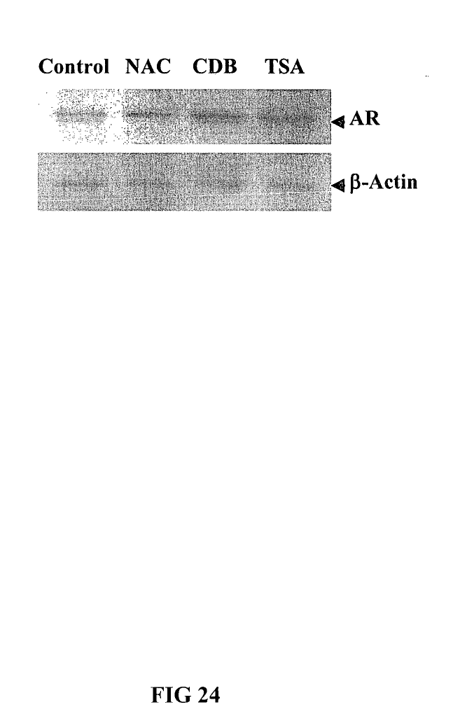

Since AR plays a critical role in antiproliferation of endometrial cells, we also measured protein expression of AR in cells treated with TSA, CDB, and NAC for 16 hrs. Western blot analysis demonstrated that AR protein levels were increased in all three treatments as compared with the control group, with the TSA group showing the highest expression, followed by CDB and NAC (FIG. 24). The result was in broad agreement with the mRNA expression levels. The effects of TSA, CDB, NAC and ICI on protein expression of ERα and ERβ were similar to the control group (FIG. 18).

Thus, it is contemplated that TSA, an HDACI, inhibits cell proliferation in endometrial cells in vitro. More intriguingly, TSA has better long-term suppression effect than CDB and NAC, the two classes of compounds that hold promise in treating endometriosis.

Possible Mechanisms of Antiproliferative Effects Induced by TSA and CDB

While the mechanism(s) of the antiproliferative effects of PA and SPRM are still not well-understood, our gene and protein expression studies provided some clues. For CDB and NAC, Fas expression was upregulated 8 h after the treatment while, interestingly, FasL expression was also increased considerably. In cells treated with TSA, there was a ˜25 fold increase in FasL expression 18 h after the treatment as compared with the controls. In all three groups, AR, PR-A, and PR-B expression all increased, with the increase in PR-B being the most pronounced (FIG. 16). This seems to support the speculation that the action of SPRM is through PR-B. Protein expression analysis revealed that the AR expression increased substantially in all groups, with TSA and CDB being the most prominent. This result is consistent with the reports on upregulation of AR in endometrium of non-human primates (Brenner et al. 2003 Steroids 68:1033-9) and women (Narvekar et al. 2004 J Clin Endocrinol Metab 89:2491-7) treated with SPRM. It also agrees with the reports that AR activity is down-regulated by HDACI in a deacetylase-dependent manner (Gaughan et al. 2002 J Biol Chem 277:25904-13), suggesting that inhibition of HDAC1 activity through TSA upregulates AR gene and protein expression, very likely by opening up chromatin structures. Since exogenous androgen can have inhibitory effects on the female reproductive tract and androgens can act as antiestrogens in estrogen treated cells (Lovely et al. 2000 J Steroid Biochem Mol Biol 74:235-41), the upregulation of AR and PRs contributes, at least in part, to the inhibition of cell proliferation.

HDACIs and RA as a Promising Therapeutics for Endometriosis

The potent and long-lasting antiproliferative activity induced by TSA strongly suggests that histone deacetylase inhibitors (HDACIs) are a promising therapeutics for endometriosis. It should be noted that TSA is only one member of an expanding family of HDACIs, which also includes valproic acid (VPA), an established drug for use in the treatment of epilepsy. It is interesting to note that there has been a long-standing debate regarding whether taking VPA is associated with hyperandrogenism and polycystic ovary syndrome. Our results suggest it is likely that VPA, as an HDACI, may suppress endometrial proliferation through upregulation of AR just as TSA.

As with any drug, they may still have undesirable side effects, as evidenced by possible causal relationship between taking VPA and hyperandrogenism and amenorrhea. Unlike any existing drug or other promising compounds, however, HDACIs have the potential to reprogram epigenetic regulatory machinery through targeting HDACIs, which in turn remodels chromatins. This is due to their ability to demethylate methylated genes in conjunction with other demethylation agents. This is particularly relevant since there is evidence for aberrant methylation at PR-B in endometriosis, which may be responsible for progestin resistance in endometriosis. This is also based principally on the fact that steroid receptors (which include ERs, PRs, and AR) of the nuclear receptor superfamily of transcription factors depend on activation by an appropriate ligand for the recruitment of histone acetylase- and HDAC-associated co-regulators. It is widely believed that the tissue-, gene-, and cell-specificity of selective receptor modulators is due to the relative balance of coactivator and corepressor expression with a given target tissue, or gene, or cell. The intimate relationship between chromatin remodeling and nuclear receptor regulation has important implications to the understanding of the pathogenesis of endometriosis, since it has been well regarded that endometriosis is a hormonal disease, with dysregulated steroid biosynthesis and metabolism. Nonetheless, we still know very little about the mechanisms of antiproliferation induced by TSA.

### Example 5

### Use of Valproic Acid to Treat Adenomyosis or Endometriosis

Briefly, adenomyosis, defined as the presence of heterotopic endometrial glands and stroma in the myometrium with adjacent smooth muscle hyperplasia, is a common gynecologic disease with a poorly understood pathogenesis and is responsible for menorrhagia, dysmenorrhea, and subfertility (Farquhar C, et al. Medical and surgical management of adenomyosis. Best Pract Res Clin Obstet Gynaecol 2006). Until recently, the diagnosis of adenomyosis is made only after hysterectomy (Lone F W, et al. Adenomyosis: not such an elusive diagnosis any longer. J Obstet Gynaecol 2006; 26(3):225-8), but now moderate to severe degrees of adenomyosis can be diagnosed with a fair degree of reliability by ultrasound or magnetic resonance imaging (Peric H, Fraser IS. The symptomatology of adenomyosis. Best Pract Res Clin Obstet Gynaecol 2006). Although the disease is hormone-sensitive, progestogenic agents are ineffective, and GnRH agonists induce suppression of adenomyosis yet their use is restricted by short duration (Bergeron C, et al. Pathology and physiopathology of adenomyosis. Best Pract Res Clin Obstet Gynaecol 2006). In addition, the symptoms reappear after discontinuation of GnRH agonists therapy (Grow D R, et al. Treatment of adenomyosis with long-term GnRH analogues: a case report. Obstet Gynecol 1991; 78(3 Pt 2):538-9).

Adenomyosis and endometriosis share a great deal of similarities—in terms of definition, estrogen dependency, and symptomology—and the former previously was used to be considered a variant of the latter (Ferenczy A. Pathophysiology of adenomyosis. Hum Reprod Update 1998 4(4):312-22). Converging evidence that endometriosis, defined as the presence of heterotopic endometrial glands and stroma outside of the uterus, is an epigenetic disease (Wu Y, et al. Aberrant expression of deoxyribonucleic acid methyltransferases DNMT1, DNMT3A and DNMT3B in women with endometriosis. Fertil Steril 2006; In press) with aberrant methylation (Wu Y, et al. Aberrant methylation at HOXA10 may be responsible for Its aberrant expression in the endometrium of patients with endometriosis. Am J Obstet Gynecol 2005 192; and Wu Y, et al. Promoter hypermethylation of progesterone receptor isoform B (PR-B) in endometriosis. Epigenetics 2006; 1(2):106-111) and may thus be amenable by demethylation agents and histone deacetylase inhibitors (HDACIs) (Szyf M. Therapeutic implications of DNA methylation. Future Oncol 2005; 1(1):125-35).

Indeed, it has been recently reported that trichostatin A (TSA), a potent HDACI, suppresses proliferation of endometrial stromal cells (Wu Y, Guo SW. Inhibition of Proliferation of Endometrial Stromal Cells By Trichostatin A, RU486, CDB-2914, N-acetylcysteine, and ICI 182780. Gynecol Obstet Invest 2006; 62(4):193-205). Further research indicates that endometriotic cells are in fact over 100 fold more sensitive than endometrial cells to TSA treatment (Wu and Guo, unpublished observation). In addition, TSA suppresses IL-1β-induced COX-2 expression (Wu Y, Guo SW. Suppression of IL-1β-induced COX-2 expression by trischotatin A (TSA) in human endometrial stromal cells. Eur J Obstet Gynecol Reprod Biol 2007 Feb. 10; [Epub ahead of print]), and both TSA and valproic acid (VPA) induce cell cycle arrest in endometrial cells. (Wu Y, Guo SW. Histone deacetylase inhibitors trichostatin A and valproic acid induce cell cycle arrest and p 21 expression in immortalized human endometrial stromal cells. Eur J Obstet Gynecol Reprod Biol. 2007 Mar. 19; [Epub ahead of print]).

Some circumstantial evidence suggests that adenomyosis may also be an epigenetic disease just like endometriosis. For example, a recent study finds that retinoblastom (pRb) is undetected in adenomyosis (Goumenou A G, Matalliotakis I M, Tzardi M, Fragouli I G, Mahutte N G, Arici A. p 16, retinoblastoma (pRb), and cyclin D1 protein expression in human endometriotic and adenomyotic lesions. Fertil Steril 2006; 85 Suppl 1: 1204-7), which may result from pRb promoter hypermethylation. There is a compelling reason to believe that HDACIs may be a promising class of compounds for treating endometriosis and perhaps adenomyosis as well.

VPA is a specific and potent HDACI (Gottlicher M, et al. Valproic acid defines a novel class of HDAC inhibitors inducing differentiation of transformed cells. Embo J 2001; 20(24):6969-78) with known and favorable pharmacokinetic properties. It has been used safely for over two decades to treat epilepsy and, more recently, bipolar disorders. Following on the heels of the discovery that endometriosis is an epigenetic disease, we conducted a pilot study on the off-label use of valproic acid to treat adenomyosis. We found that by the end of the 3-month treatment, all three recruited patients reported complete disappearance of dysmenorrhea with an average reduction of uterus size by 33%.

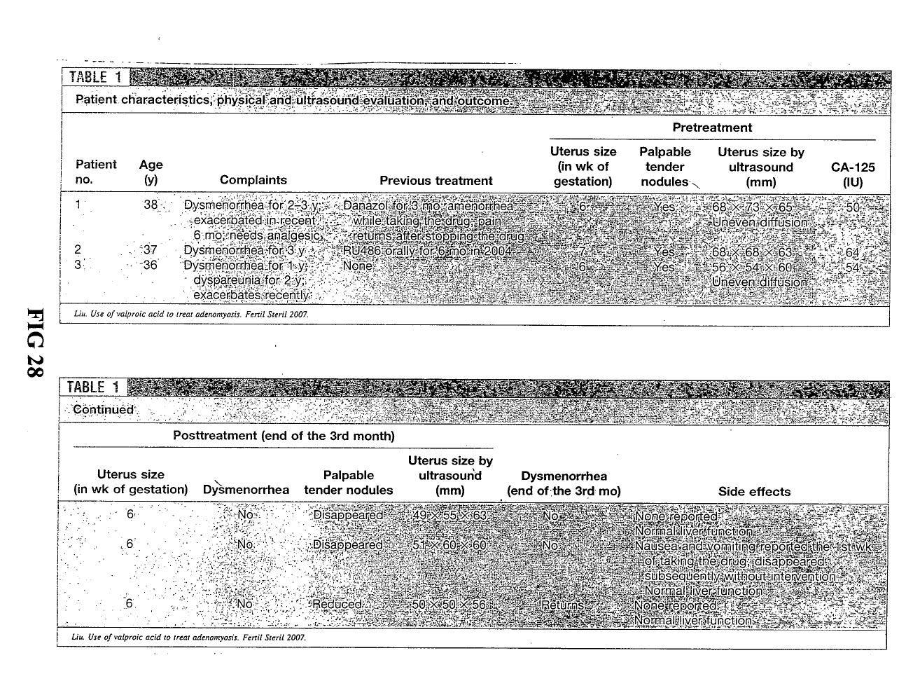

After informed consent, we recruited three patients diagnosed with adenomyosis (FIG. 28). All three patients had moderate to severe dysmenorrehea. They were all married and parous, and had normal menstrual cycles (28-30 days). They all had normal hepatic and renal function, as defined by serum transaminases <1.5×upper limit of normal (ULN), bilirubin <1.5×ULN, and creatinine <2.0×ULN. They were not pregnant or breast feeding and had no intention to get pregnant, and did not have any psychiatric condition considered to compromise full compliance with the study or severe concomitant medical disorder, significant cardiovascular disease, major thromboembolic event in the last 6 months, or grade 3 or 4 bleeding. During the recruitment, all patients were offered the option of hysterectomy or the chance to try an experimental drug. They all chose to try the drug and were all given the full explanation of the experimental nature of this study and possible risks associated with it. Approval for this pilot study was obtained from the local ethics committee at Shanghai OB/GYN Hospital, Fudan University.

The diagnosis of adenomyosis of all three patients was made based on symptomology, pelvic examination and vaginal ultrasound evaluation by experienced physicians (FIG. 28). All patients received VPA capsules orally (Depakine®, Sanofi-Aventis) starting at the 5^{th} day of the menstrual cycle, with 500 mg b.i.d. for the first week and t.i.d. thereafter when no serious adverse effect was reported. The treatment was terminated at the end of the 3^{rd} month after taking the drug and the patients were followed up for an additional 3 months. During treatment, clinical signs, symptoms and ultrasound evaluation were closely monitored. Blood counts and biochemistries including liver function tests, ammonium and coagulation studies were performed monthly.

Two months after taking VPA, 2 patients reported complete relief of dysmenorrehea with no need for pain-killers, and 1 reported much relieved pain. Three months after taking the drug, all three patients reported complete relief of pain without any need for pain-killers. Three months after the discontinuation of the treatment, 2 patients reported disappearance of dysmenorrehea, but 1 reported the return of the same symptom.

Pelvic and ultrasound examinations one month after taking VPA indicated no change in uterus size or other conditions (data not shown). At the end of the 3^{rd} month, the physical examination indicated slight reduction in uterus size in one patient but no change in the other two (FIG. 28). However, the ultrasound evaluation indicated that the uterus size in volume was reduced by 15.5%, 60.6%, and 22.8%, respectively, in the three patients (FIG. 28), with an average reduction of ⅓. In addition, the palpable tender nodular in the cul-de-sac disappeared in two cases (JWH and LYL) and decreased significantly in 1 case (LMH). Taken together, more objective measurements appeared to corroborate with the relief of pain as reported by the patients.

The serum level of testosterone in the three patients was within the normal range at the end of three month after taking VPA (0.29, 0.35, and 0.40 μg/ml, respectively). The length of the menstrual cycle was increased in two cases (JWH and LYL) to 40-45 days, but no such change was observed in one case (LMH).

VPA appeared to be well tolerated as reported (Tunnicliff G. Actions of sodium valproate on the central nervous system. J Physiol Pharmacol 1999; 50(3):347-65). Only one case (LYL) reported transient nausea and vomiting one week after taking the drug, which disappeared subsequently without any intervention. In all cases, liver function and other biochemistries remained normal.

Thus, to applicants best knowledge, this is the first report of rather encouraging results on the use of VPA to treat adenomyosis, which seems to suggest the feasibility and certain efficacy of VPA in treating this disease that is known to be challenging for medical treatment. Granted, this study is not a randomized trial, and has a very small sample size and no controls. In addition, the diagnosis was not based on histologic or laparoscopic findings. The 2 cases out of 3 reporting disappearance of dysmenorrhea 3 months after termination of the 3-month VPA treatment could well be attributed to possible placebo effect. Nonetheless, the results are still very encouraging because all three patients reported complete relief of dysmenorrehea by the end of the three-month treatment, an event that could have been accounted for only if the placebo effect were at least 37% (0.37^{3>0.05}). The purported placebo effect also had to be somewhat long-lasting: after 3 months off the drug, 2 out of 3 patients still remained pain-free. Further, given the proven ability of TSA and VPA in suppressing proliferation of endometrial and endometriotic cells in vitro (11), the consistent and considerable reduction (˜33%) in uterus size across all three patients suggests that the relief of dysmenorrehea in the two patients is unlikely due entirely to placebo effect.

Although the exact mechanisms of alleviating pain by VPA are far from clear, it is possible that VPA treatment may alleviate pelvic pain by inhibiting proliferation, inducing apoptosis and cell cycle arrest, suppressing COX-2 expression, and inhibiting angiogenesis, adhesion, invasion and the production of proinflammatory cytokines and chemokines.

The suppression of production of proinflammatory cytokines and chemokines alone as a result of VPA treatment may help alleviate pain, since the crosstalk between chemokines and neuronal receptors that regulate pain (Johannessen CU. Mechanisms of action of valproate: a commentatory. Neurochem Int 2000; 37(2-3):103-10) may contribute, at least in part, to the adenomyosis-associated pain. It has been reported RANTES (CCL5) and MIP-1α(CCL3) desensitize μ-opioid receptors (Zhang N, et al. Oppenheim JJ. Proinflammatory chemokines, such as C-C chemokine ligand 3, desensitize mu-opioid receptors on dorsal root ganglia neurons. J Immunol 2004; 173(1):594-9) but sensitize the heat- and capsaicin-gated ion channel transient receptor potential vanilloid 1 (TRPV1) (Zhang N, et al. A proinflammatory chemokine, CCL3, sensitizes the heat- and capsaicin-gated ion channel TRPV1. Proc Natl Acad Sci USA 2005; 102(12):4536-41), which are likely to contribute pain in adenomyosis and endometriosis. The increased production of proinflammatory chemokines activates their corresponding receptors, which in turn down-regulates the analgesic functions of opioid receptors and up-regulates TRPV1, resulting enhanced perception of pain at inflammatory sites. It is also interesting to note that cytokine TNFα also plays a critical role in the development and maintenance of neuropathic pain (Woolf C J, et al. Cytokines, nerve growth factor and inflammatory hyperalgesia: the contribution of tumour necrosis factor alpha. Br J Pharmacol 1997; 121(3):417-24). By suppressing the production of proinflammatory cytokines and chemokines, VPA may alleviate pelvic pain.

Although pelvic examination suggests no change in uterus size, a more objective evaluation by ultrasound nonetheless indicates reduction by as much as 60.8% three months after taking VPA. A more dramatic reduction in uterus size could be achieved through longer use of VPA since GnRH agonists are typically given for 6 months to relieve dysmenorrhea and menorrhagia (Ferenczy A. Pathophysiology of adenomyosis. Hum Reprod Update 1998; 4(4):312-22). In addition, the combination of VPA with retinoic acid may also induce further reduction in uterus size since retinoic acid appears to synergize with VPA in suppression of proliferation of endometrial cells (Wu and Guo, unpublished observation).

Applicants note that the subjective feeling of pelvic pain or lack of it actually is quite important to the well-being of the patient. It was the accelerated return of pelvic pain reported by the patients, not the increase in lesion size or number of lesions, that prompted the termination of a recent phase II trial on the use of raloxifene to treat endometriosis (Stratton P., et al. Raloxifene accelerates the return of chronic pelvic pain from endometriosis after surgical treatment [Abstract]. In: The IX World Congress on Endometriosis. Maastricht, Netherlands; 2005) despite all encouraging results from animal studies (Yao Z. et al., Validation of rat endometriosis model by using raloxifene as a positive control for the evaluation of novel SERM compounds. J Invest Surg 2005; 18(4):177-83).

In view of the cumulating evidence that endometriosis is an epigenetic disease and the indication it may be rectified by HDACIs, our encouraging results on the pilot study of the off-label use of VPA to treatment adenomyosis should be greeted with guarded enthusiasm. Obviously, whether VPA or other HDACIs may be truly efficacious in treating adenomyosis and/or endometriosis should await future controlled randomized clinical trials with optimal treatment duration, sufficient sample sizes, and more objective quantification. If proven efficacious, VPA may be a much cheaper alternative to GnRH agonist therapy, possibly more efficacious and potent due to its potential to rectify epigenetic aberrations yet with less and milder side effects.

All of the documents cited herein are incorporated by reference here in their entirety).

Unless otherwise defined, all technical and scientific terms used herein have the same meaning as commonly understood by one of ordinary skill in the art to which this invention belongs. Although suitable methods and materials for the practice or testing of the present invention are described below, other methods and materials similar or equivalent to those described herein, which are well known in the art, can also be used.

We claim:

1. A method of diagnosing endometriosis in a test subject, the method comprising the steps of:
a) determining the methylation state of a progesterone receptor-B (PR-B) gene promoter, or a HOXA10 gene in a biological sample from the test subject;
b) comparing the methylation state of each gene in step a) with the methylation state of each gene in a normal subject not having endometriosis, wherein a hypermethylated state in a region within either gene or associated regulatory regions is indicative of the test subject having endometriosis.

2. The method of claim 1 wherein the endometriosis is resistant to progestin or progesterone therapy.

3. The method of claim 1 wherein the biological sample is menstrual fluid.

4. A method for detecting in a subject cell proliferation of endometriotic cells associated with endometriosis, the method comprising the steps of:
a) obtaining a biological sample from the subject;
b) determining the methylation state of a progesterone receptor-B (PR-B) gene, or a HOXA10 gene in the subject's sample; and
c) identifying aberrant methylation of regions of the gene or regulatory region, wherein aberrant methylation is identified as being different when compared to the same regions of the gene or associated regulatory region in a subject not having the cellular proliferative disorder, thereby detecting in the subject a cellular proliferative disorder associated with endometriosis; wherein aberrant methylation comprises hypermethylation when compared to the same regions of the gene or associated regulatory regions in a subject not having the cellular proliferative disorder.

5. The method of claim 4 wherein the regulatory region exhibiting hypermethylation is the PR-B promoter.

6. The method of claim 4 wherein the gene exhibiting aberrant methylation is the HOXA10 gene.

7. The method of claim 4 wherein the endometriosis is resistant to progestin or progesterone therapy.

8. The method of claim 4 wherein the biological sample is menstrual fluid.

9. The method of claim 4 further comprising the step of inhibiting proliferation of the endometriotic cells by administering to the subject an effective amount of a histone deacetylase inhibitor (HDACI).

10. The method of claim 9 wherein the inhibitor is valproic acid or trichostatin A.

11. The method of claim 9 further comprising co-administering an effective amount of a demethylation agent.

12. The method of claim 11 wherein the demethylation agent is 5-azacytidine.

13. The method of claim 9 further comprising co-administering an effective amount of retinoic acid (RA), suitably 9-cis RA or all-trans RA.

14. A method for diagnosing endometriosis in a test subject, the method comprising the steps of:
a) assaying a biological sample obtained from the test subject to determine the expression level of any of the genes comprising progesterone receptor-B (PR-B), HOXA10, and DNA methyltransferase (DNMT), or a combination thereof in the sample; and
b) comparing the expression level for any of the genes in step a) to the expression level for the same gene in a normal subject not having endometriosis; wherein decreased expression level of the PR-B and HOXA10 or an increased expression level of the gene coding for DNMT compared to the expression level of the same gene in a normal subject is indicative of the test subject having endometriosis.

15. The method of claim 14 wherein the DNMT genes include DNMT1, DNMT3A and DNMT3B.

16. The method of claim 14 wherein the method includes a genetic assay.

17. The method of claim 14 wherein the method includes an immunoassay.

18. The method of claim 14 wherein the endometriosis is resistant to progestin or progesterone therapy.

19. The method of claim 14 wherein the biological sample is menstrual fluid.

20. A method for diagnosing a subject's predisposition to endometriosis, the method comprising the steps of:
a) taking menstrual blood sample or endometrial cells from the subject;
b) determining the expression pattern of at least one gene selected from the group consisting of PR-B, HOXA10, and the gene coding for DNA methyltransferase in the cells associated with endometrial pathology to determine if the subject has an altered expression pattern in the gene; and
c) diagnosing the subject as predisposed to endometriosis if the expression pattern of the selected gene is altered in the subject as compared to a cell from a normal, non-endometriotic subject.

21. The method of claim 20 wherein the DNA methyltransferase (DNMT) is DNMT1, DNMT3A, and DNMT3B, and wherein DNMT is overexpressed relative to a cell from a normal, non-endometriotic subject.

22. A method of selecting a therapy for treating endometriosis, the method comprising the step of:
a) assaying a biological sample for the presence of endometriotic cells with at least one biomarker associated with endometrial pathology;
b) detecting at least one biomarker, wherein the biomarker includes of an increase in DNA methyltransferase (DNMT) expression level, hypermethylation of PR-B promoter or aberrant methylation of the HOXA10 gene in such cells relative to a control sample; and
c) selecting a therapy according to the results of the detection, wherein the therapy may include administering a demethylation agent alone or in combination with histone deacetylase inhibitors.

23. A biomarker predictive of endometriosis, wherein the biomarker is a DNA methyltransferase (DNMT), preferably DNMT1, DNMT3A, and DNMT3B having an increased expression level relative to a control sample, wherein the endometriosis is ectopic or eutopic.

24. A DNA biomarker predictive of endometriosis, wherein the biomarker is a hypermethylated progesterone receptor promoter, preferably PR-B isoform in a subject with endometriosis relative to a control subject.

25. A DNA biomarker predictive of endometriosis, wherein the biomarker is an aberrantly methylated HOXA10 gene, or a decreased expression level of the HOXA10 gene in a subject with endometriosis relative to a control subject.

26. A method for monitoring disease progression and/or treatment efficacy and/or relapse of endometriosis, the method comprising assaying endometriotic cells for the presence of a biomarker, wherein the biomarker includes any one of hypermethylated progesterone receptor promoter, a decreased expression level of a progesterone receptor, aberrant methylation of a HOXA10 gene, decreased expression level of HOXA10 gene, and increased expression of DNA methyltransferase (DNMT), preferably DNMT1, DNMT3A, and DNMT3B relative to a control sample.

27. A method of downregulating the expression of DNA methyltransferases (DNMT) in a subject having endometriosis, the method comprising administering an effective amount of a histone deacetylase inhibitor (HDACI) to the subject, such that DNMT expression is downregulated at least about 1.5 times as much as compared with DNMT expression in a normal subject.

28. The method of claim 27 wherein the HDACI is valproic acid or derivatives and analogs thereof.

29. The method of claim 27 wherein the downregulation of DNMT results in decreased progesterone receptor (PR) gene methylation, increased PR gene expression, increased progesterone binding, and increased responsiveness to progesterone therapy relative to normal DNMT expression in a subject.

30. A method of downregulating the expression of DNA methyltransferases (DNMT) in a subject having endometriosis, the method comprising co-administering an effective amount of a histone deacetylase inhibitor (HDACI) and a demethylation agent to the subject, such that DNMT expression is downregulated, and wherein DNMT includes DNMT1, DNMT3A, and DNMT3B.

31. The method of claim 30 wherein the HDACI is valproic acid and the demethylation agent is 5-azacytidine.

32. A method of treating endometriosis by inhibiting cell proliferation of endometriotic cells in a subject, comprising administering an effective amount of a histone deacetylase inhibitor (HDACI).

33. The method of claim 32 wherein the inhibitor is valproic acid or derivatives and analogs thereof.

34. The method of claim 32 further comprising co-administering an effective amount of a demethylation agent.

35. The method of claim 34 wherein the demethylation agent is 5-azacytidine.

36. The method of claim 32 further comprising co-administering an effective amount of retinoic acid (RA), suitably 9-cis RA or all-trans RA.

37. A kit for assaying the presence of promoter hypermethylation of progesterone Receptor Isoform B (PR-B) in a biological sample relative to a control, the kit comprising at least one primer pair or one oligonucleotide capable of selectively hybridizing to the progesterone receptor isoform B (PR-B) gene.

38. The kit of claim 37 wherein the primer pair or oligonucleotide include SEQ ID Nos: 5-8 and 11-12.

39. A kit for assaying the presence of aberrant methylation in HOXA10 gene in a biological sample relative to a control, the kit comprising at least one primer pair or one oligonucleotide capable of selectively hybridizing to the HOXA10 gene.

40. The kit of claim 39 wherein the primer pair or oligonucleotide include SEQ ID Nos: 26-33 and 34-39.

41. A kit for assaying for the presence of an increased DNA methyltransferase expression level in a biological sample relative to a control, the kit comprising at least one primer pair or one oligonucleotide capable of selectively hybridizing to the DNA methyltransferase (DNMT) gene, where in the DNMTs include DNMT1, DNMT3A, and DNMT3B.

42. A kit for detecting endometriosis, comprising a kit comprising an antibody against a DNA methyltransferase (DNMT), preferably DNMT1, DNMT3A, and DNMT3B, wherein the detection is performed by an immunoassay.

43. The kit of claim 42, wherein the kit further comprises at least one reagent for performing an immunoassay, preferably an ELISA or a Western blot.
